(19) United States
(12) Patent Application Publication
(10) Pub. No.: US 2013/0237550 A1
(43) Pub. Date: Sep. 12, 2013

(54) ARYLVINYLAZACYCLOALKANE COMPOUNDS FOR CONSTIPATION

(71) Applicants: Steven M. Toler, Winston-Salem, NC (US); David Hosford, Durham, NC (US); Patrick M. Lippiello, Lewisville, NC (US); Geoffrey Charles Dunbar, Winston-Salem, NC (US); William Scott Caldwell, Winston-Salem, NC (US); Parviz Hamedani, Califon (NJ); Merouane Bencherif, Winston-Salem, NC (US)

(72) Inventors: Steven M. Toler, Winston-Salem, NC (US); David Hosford, Durham, NC (US); Patrick M. Lippiello, Lewisville, NC (US); Geoffrey Charles Dunbar, Winston-Salem, NC (US); William Scott Caldwell, Winston-Salem, NC (US); Parviz Hamedani, Califon (NJ); Merouane Bencherif, Winston-Salem, NC (US)

(73) Assignee: TARGACEPT, INC., Winston-Salem, NC (US)

(21) Appl. No.: 13/583,644

(22) PCT Filed: Mar. 4, 2011
(86) PCT No.: PCT/US11/27111

§ 371 Date: Nov. 13, 2012

Related U.S. Application Data
(60) Provisional application No. 61/312,944, filed on Mar. 11, 2010.

(51) Int. Cl. A61K 31/506; A61K 45/06

(52) U.S. Cl. CPC A61K 31/506; A61K 45/06
USPC 514/256

(57) ABSTRACT

The present invention relates to method of treating constipation and enhancing colonic motility by administration of 5-((E)-2-pyrrolidin-3-ylvinyl)pyrimidine or a pharmaceutically acceptable salt thereof.

### FIELD OF THE INVENTION

The present invention relates to methods of treating constipation and enhancing colonic motility by administration of 5-((E)-2-pyrrolidin-3-ylvinyl)pyrimidine or a pharmaceutically acceptable salt thereof.

### BACKGROUND OF THE INVENTION

Constipation, also known as costiveness, dyschezia, and dyssynergic defaecation, refers to bowel movements that are infrequent and/or hard to pass. Constipation is a common cause of painful defecation. Severe constipation includes obstipation (failure to pass stools or gas) and fecal impaction (bowel obstruction). Constipation is common; in the general population incidence of constipation varies from 2 to 30%.

The Rome III criteria are widely used to diagnose chronic constipation and are helpful in separating cases of chronic functional constipation from less-serious instances. Also, the Bristol Stool Scale or Bristol Stool Chart is a medical aid designed to classify the form of human feces into seven categories. Sometimes referred to as the “Meyers Scale”, it was developed at the University of Bristol and was first published in the Scandinavian Journal of Gastroenterology in 1997. Types 1 and 2 from the Bristol Stool Chart indicate constipation. Reference is made to Lewis, S., Heaton, K. (1997), Stool Form Scale as a Useful Guide to Intestinal Transit Time. Scand. J. Gastroenterol. 32 (9): 920-4.

The causes of constipation are of two types: obstructed defecation and colonic slow transit (or hypomobility). About 50% of patients evaluated for constipation at tertiary referral hospitals have obstructed defecation. This type of constipation has mechanical and functional causes. Causes of colonic slow transit constipation include diet, hormones, side effects of medications, and heavy metal toxicity. The definition of constipation includes the following: infrequent bowel movements, typically three times or fewer per week; difficulty during defecation, straining during more than 25% of bowel movements or a subjective sensation of hard stools); or the sensation of incomplete bowel evacuation.

The causes of constipation can be further divided into congenital, primary, and secondary. The most common cause is primary and not life threatening. Causes include insufficient dietary fiber intake, inadequate fluid intake, decreased physical activity, side effects of medications, hypothyroidism, and obstruction by colorectal cancer. Primary or functional constipation is defined to be ongoing symptoms for greater than six months not due to any underlying cause such as medication side effects or an underlying medical condition. It is not associated with abdominal pain thus distinguishing it from irritable bowel syndrome. It is the most common cause of constipation. Constipation can be caused or exacerbated by a low fiber diet, low liquid intake, or dieting. Many medications have constipation as a side effect. Some examples include: opioids, diuretics, antidepressants, antihistamines, antispasmodics, anticonvulsants, and aluminum antacids. Metabolic and endocrine problems which may lead to constipation include: hypercalcemia, hypothyroidism, diabetes mellitus, cystic fibrosis, and celiac disease. Constipation is also common in individuals with muscular and myotonic dystrophy. Lastly, several reports note a link between smoking cessation and the onset of constipation. See, e.g., Lagrue et al., Stopping Smoking and Constipation, as translated from the original French, Addiction, November 2003, 98(11): 1563-7.

Constipation has a number of structural (mechanical, morphological, anatomical) causes, including: spinal cord lesions, Parkinson's, colon cancer, anal fissures, proctitis, and pelvic floor dysfunction.

Constipation also has functional (neurological) causes, including anismus, descending perineum syndrome, and Hirschsprung's disease. In infants, Hirschsprung's disease is the most common medical disorder associated with constipation. Anismus occurs in a small minority of persons with chronic constipation or obstructed defecation.

Chronic constipation occurs in from 12% to 20% of the US population. One definition of chronic constipation is three or fewer bowel movements per week for three months or more in a year. Associated medical costs, based upon estimates of 2.5 million physician visits and limited treatment options, which are limited largely to over-the-counter products, are believed over $7 billion per year.

Irritable Bowel Syndrome (IBS) represents a therapeutic area with unmet medical need with respect to currently available treatment options. Past research has focused on targeting serotonin and its role in the regulation of gastric motility and secretions. Progress has been slowed by the cardiovascular dose-limiting side effects of available treatment options.

IBS involves daytime abdominal pain, bloating and discomfort and altered bowel habits characterized by predominance of one of the following: Constipation (IBS-C); Diarrhea (IBS-D); and both alternating (IBS-A or Alternating IBS). IBS afflicts 12% of adults in the US, and has an incidence among women twice as high as men. See, Mertz, H. Irritable bowel syndrome. N. Engl. J. Med. 349, 2136-2146 (2003). The current drug development paradigms have focused on medications that alter the action of serotonin in the colon. Research has revealed that serotonin (5-HT) is involved in mediating intestinal motility, controlling intestinal secretion in the GI tract, and adjusting sensation in the bowels. To date, interest in this approach has focused on 5-HT₃ and 5-HT₄ receptors. Adverse 5-HT pharmacology concerns including adverse cardiovascular side-effects temper the interest in the 5-HT therapy.

Chronic idiopathic constipation (CIC) is a diagnosis given to individuals who have a healthy bowel and suffer from chronic constipation, but whose symptoms are not relieved through standard treatment. CIC is differentiated from constipation predominant irritable bowel syndrome (IBS-C) by the lack of pain as a primary symptom.

A variety of metanicotine analogs have been proposed for use in treating a variety of disorders, including IBS. See, for example, U.S. Pat. No. 7,098,331, and published PCT WO 2010/065443, the contents of which are hereby incorporated by reference. Some of these compounds suffer from deleterious effects upon administration, for example, emesis and nausea as a result of drug exposure in the upper GI tract.

It would be advantageous to provide new treatments, especially treatments which target nicotinic receptors as an alternative to the 5-HT approach for constipation in its various manifestations. The present invention provides such compositions and methods.

### SUMMARY

One aspect of the present invention includes methods, uses, and compositions for use for 5-((E)-2-pyrrolidin-3-ylvinyl)pyrimidine or a pharmaceutically acceptable salt thereof.

One aspect of the present invention includes a method for relieving constipation comprising administration of (R)-5-((E)-2-pyrrolidin-3-ylvinyl)pyrimidine or a pharmaceutically acceptable salt thereof. One embodiment of the present invention includes treating constipation wherein the source of the constipation is: gastrointestinal, including but not limited to irritable bowel syndrome, including IBS-A and IBS-C, acute or chronic idiopathic constipation, colon cancer, or ileus paralyticus; endrocrinological, including but not limited to pregnancy or hypothyroidism; neurological, including but mot limited to Parkinson's Disease, multiple schlerosis, or depression; iatrogenic, including but not limited to treatment with opiates, antidepressants, antacid medicines, or iron supplements; associated with an eating disorder, such as anorexia or bulimia as well as eating disorders associated with stress, travel, and dietary changes; or related to an injury, including surgery, spinal cord injury, autonomic dysfunction, paraplegia, age, or long-term patient care.

Another aspect of the present invention includes a method for treating constipation associated with a gastrointestinal disorder comprising administration of (R)-5-((E)-2-pyrrolidin-3-ylvinyl)pyrimidine or a pharmaceutically acceptable salt thereof. In one embodiment, the gastrointestinal disorder is irritable bowel syndrome, constipation predominant irritable bowel syndrome, alternating irritable bowel syndrome, chronic idiopathic constipation, acute constipation, drug-induced constipation, colonic disorders, colon cancer, or ileus paralyticus. As a further embodiment, the drug-induced constipation preferably is opioid-induced constipation, antidepressant-induced constipation, antacid-induced constipation, or iron supplement-induced constipation.

Another aspect of the present invention includes a method for treating constipation from an endocrinological source comprising administration of (R)-5-((E)-2-pyrrolidin-3-ylvinyl)pyrimidine or a pharmaceutically acceptable salt thereof. In one embodiment, the endocrinological source is pregnancy or hypothyroidism.

Another aspect of the present invention includes a method for treating constipation associated with a neurological condition comprising administration of (R)-5-((E)-2-pyrrolidin-3-ylvinyl)pyrimidine or a pharmaceutically acceptable salt thereof. In one embodiment, the neurological condition is Parkinson's Disease, multiple sclerosis, or depression.

Another aspect of the present invention includes a method for treating constipation associated with an eating disorder comprising administration of (R)-5-((E)-2-Pyrrolidin-3-ylvinyl)pyrimidine or a pharmaceutically acceptable salt thereof.

Another aspect of the present invention includes a method for treating constipation associated with surgery, spinal cord injury, autonomic dysfunction, paraplegia, age, or long-term patient care comprising administration of (R)-5-((E)-2-pyrrolidin-3-ylvinyl)pyrimidine or a pharmaceutically acceptable salt thereof.

As another aspect of the present invention, the method, use, or composition for use of the present invention further comprises administration of one or more additional therapeutic agent.

Another aspect of the present invention includes a method for enhancing colonic motility comprising administration of (R)-5-((E)-2-pyrrolidin-3-ylvinyl)pyrimidine or a pharmaceutically acceptable salt thereof.

Another aspect of the present invention includes a method for treating a mammal in need thereof with (R)-5-((E)-2-pyrrolidin-3-ylvinyl)pyrimidine or a pharmaceutically acceptable salt thereof to relieve constipation. Such treatment includes human pharmaceutical use as well as veterinary use, including but not limited to treatment of rabbits, cats, dogs, cows, horses, or other animals.

The present invention includes pharmaceutical presentations, including enteric presentations, of 5-((E)-2-pyrrolidin-3-ylvinyl)pyrimidine or a pharmaceutically acceptable salt thereof. One aspect of the present invention includes an enteric oral pharmaceutical product comprising (R)-5-((E)-2-pyrrolidin-3-ylvinyl)pyrimidine or a pharmaceutically acceptable salt thereof. In one embodiment, the product is a tablet. In one embodiment, the product is a capsule or a core sheathed in an annular body. In one embodiment, the product comprises an enteric coating which is essentially not dissolvable in the stomach surrounding a core which comprises the active ingredient. In one embodiment, the product contains said (R)-5-((E)-2-pyrrolidin-3-ylvinyl)pyrimidine or a pharmaceutically acceptable salt thereof as the sole active ingredient. In one embodiment the product comprises: (a) a core consisting of (R)-5-((E)-2-pyrrolidin-3-ylvinyl)pyrimidine or a pharmaceutically acceptable salt thereof and one or more pharmaceutical excipients—either in a unitary or multiparticulate presentation; (b) an optional separating layer; (c) an enteric layer comprising polymethacrylates and a pharmaceutically acceptable excipient; and (d) an optional finishing layer. In one embodiment of the multiparticulate presentation, the core comprises an inert bead on which the (R)-5-((E)-2-pyrrolidin-3-ylvinyl)pyrimidine or a pharmaceutically acceptable salt thereof is deposited as a layer comprising said one or more pharmaceutical excipients. In one embodiment, the product contains about 0.5 to 50 milligrams of (R)-5-((E)-2-pyrrolidin-3-ylvinyl)pyrimidine or a pharmaceutically acceptable salt thereof.

One aspect of the present invention includes an oral pharmaceutical dosage form comprising (R)-5-((E)-2-pyrrolidin-3-ylvinyl)pyrimidine or a pharmaceutically acceptable salt thereof and adapted to retard or inhibit the release of (R)-5-((E)-2-pyrrolidin-3-ylvinyl)pyrimidine or a pharmaceutically acceptable salt thereof in the stomach. In one embodiment, the oral pharmaceutical dosage form is a tablet, a capsule, or a core sheathed in an annular body. In one embodiment, the pharmaceutical dosage form is a tablet. In one embodiment, the pharmaceutical dosage form is a capsule. In one embodiment, the pharmaceutical dosage form includes an enteric coating.

One aspect of the present invention includes a method for treating irritable bowel syndrome comprising administration of an enteric coated pharmaceutical dosage form of (R)-5-((E)-2-pyrrolidin-3-ylvinyl)pyrimidine or a pharmaceutically acceptable salt thereof. In one embodiment, the irritable bowel syndrome is constipation predominant irritable bowel syndrome. In one embodiment, the pharmaceutical dosage form of (R)-5-((E)-2-pyrrolidin-3-ylvinyl)pyrimidine or a pharmaceutically acceptable salt thereof comprises encapsulated multi-particulate pellets. In one embodiment, the administration is about 0.5 to 50 milligrams of (R)-5-((E)-2-pyrrolidin-3-ylvinyl)pyrimidine or a pharmaceutically acceptable salt thereof. In one embodiment, the enteric coating comprises a polymethacrylate. In one embodiment, the (R)-5-((E)-2-pyrrolidin-3-ylvinyl)pyrimidine or a pharmaceutically acceptable salt thereof is delivered to the lower GI tract.

One aspect of the present invention includes a method of delivering (R)-5-((E)-2-pyrrolidin-3-ylvinyl)pyrimidine or a pharmaceutically acceptable salt thereof selectively to the lower GI tract comprising a pharmaceutical dosage form having an enteric coating.

The scope of the present invention is described in further detail herein and includes all combinations of aspects and embodiments.

### BRIEF DESCRIPTION OF THE FIGURES

FIG. 1 is a bar graph representation of SBM observed in IBS-C subjects. The left portion of the figure demonstrates objective count of SBM on a weekly basis. The right portion of the figure illustrates the efficacy of Compound A compared to placebo across the entire 4 week treatment period.

FIG. 2 is a bar graph representation of a relative comparison of SBM across several therapeutics. Compound A was compared with existing and proposed therapies, namely Tegaserod (previously sold under the brand name Zelnorm®, currently withdrawn), Lubiprostone (sold under the trade name Amitiza®), and Linaclotide (currently in Phase III clinical trials), as well as placebo in each case. As illustrated, Compound A compares favorably in SBM at week 4 in subjects with IBS-C.

### DETAILED DESCRIPTION

Provided herein are formulations engineered to initiate drug release in the middle to lower portions of the small intestine, with a delayed release time of greater than, for example, approximately 1 hour, 1.25 hours, 1.5 hours, 1.75 hours or 2 hours after dosing. Such pharmaceutical formulations are manufactured in such a way that the product passes unchanged through the stomach of the patient, and dissolves and releases the active ingredient when it leaves the stomach and enters the middle and lower portions of the small intestine. Such formulations can be in tablet or pellet form, where the active ingredient is in the inner part of the tablet or pellet and is enclosed in a film or envelope, the “enteric coating,” which is insoluble in acid environments, such as the stomach, but is soluble in near-neutral environments such as the small intestine.

As used herein, all expressions of percentage, ratio, proportion and the like, will be in weight units unless otherwise stated. Expressions of proportions of the enteric product will refer to the product in dried form, after the removal of the water in which many of the ingredients are dissolved or dispersed. The term “sugar” refers to a sugar other than a reducing sugar. A reducing sugar is a carbohydrate that reduces Fehling's (or Benedict's) or Tollens' reagent. All monosaccharides are reducing sugars as are most disaccharides with the exception of sucrose. One common binding or filling agent is lactose. This excipient is particularly useful for tablets since it compresses well, is both a diluent and binder, and is cheap. However, it is a reducing sugar and it may be that the active ingredient interacts with lactose both at room temperature and under accelerated stability conditions (heat). Therefore, avoidance of lactose and other reducing sugars from formulations comprising the active ingredient may be important. As discussed below, sucrose is a particular sugar.

In a particular enteric product, a core of active is surrounded by an enteric coat and formed into a pellet. The pellets can then be loaded into gelatin capsules. The various components and layers of the pellet will be individually discussed as follows, together with the methods of adding the different ingredients to build up the pellet.

### I. Compound

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or exipients. Pharmaceutical compositions preferably exhibit a degree of stability to environmental conditions so as to make them suitable for manufacturing and commercialization purposes.

As used herein, the terms “effective amount”, “therapeutic amount”, or “effective dose” refer to an amount of the compound of the present invention sufficient to elicit the desired pharmacological or therapeutic effects, thus resulting in effective prevention or treatment of a disorder. Prevention of the disorder may be manifested by delaying or preventing the progression of the disorder, as well as the onset of the symptoms associated with the disorder. Treatment of the disorder may be manifested by a decrease or elimination of symptoms, inhibition or reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds are required to be administered in an amount of less than 5 mg/kg of patient weight. Often, the compounds may be administered in an amount from less than about 1 mg/kg patient weight to less than about 100 μg/kg of patient weight, and occasionally between about 10 μg/kg to less than 100 μg/kg of patient weight. The foregoing effective doses typically represent that amount administered as a single dose, or as one or more doses administered over a 24 hours period. For human patients, the effective dose of the compounds may require administering the compound in an amount of at least about 1 mg/24 hr/patient, but not more than about 1000 mg/24 hr/patient, and often not more than about 500 mg/24 hr/patient. As will be noted in further detail below, a total dose of 5 mg (or <100 μg/kg) demonstrates efficacy. One likely efficacious dose for the present invention likely is between about 10 μg/kg and about 100 μg/kg.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons,). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art, but are not mentioned in greater detail.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted. Although only one delocalized resonance structure may be depicted, all such forms are contemplated within the scope of the invention.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### II. General Synthetic Methods

(R)-5-((E)-2-pyrrolidin-3-ylvinyl)pyrimidine or a pharmaceutically acceptable salt thereof can be prepared by a variety of synthetic strategies which will be apparent to those of skill in the art. In one aspect, (R)-5-((E)-2-pyrrolidin-3-ylvinyl)pyrimidine or a pharmaceutically acceptable salt thereof can be obtained using the palladium catalyzed coupling reaction of a aryl halide with an vinylpyrrolidine compound (see, for instance U.S. Pat. No. 7,098,331 and published PCT application WO 10/065,443). Thus reaction of a suitably N-protected 3-vinylpyrrolidine with a 5-bromopyrimidine in the presence of palladium(II) acetate, triphenylphosphine and triethylamine yields an N-protected (E)-(2-pyrrolidin-3-ylvinyl)pyrimidine. Subsequent removal of the protecting group produces (R)-5-((E)-2-pyrrolidin-3-ylvinyl)pyrimidine or a pharmaceutically acceptable salt thereof.

Methods for protection and deprotection of the pyrrolidine nitrogen are well known to those of skill in the art of synthetic chemistry and can be found in compilations, such as “Protective Groups in Organic Synthesis (2^{nd} ed.)”, by T. W. Greene and P. G. M. Wuts (Wiley-Interscience (1991)). The requisite starting materials for the coupling reaction (the heteroaryl halide and the 3-vinylpyrrolidine) can be by numerous methods. The N-protected 3-vinylpyrrolidines can be made from the corresponding N-protected 3-formylpyrrolidines by Wittig olefination using methylenetriphenylphosphorane. Other conversions of aldehydes to vinyl groups are known to those of skill in the art. The 3-formylpyrrolidine can be made in either one or two steps from the corresponding ester (e.g., N-protected alkyl pyrrolidine-3-carboxylate) by reduction with diisobutylaluminum hydride or reduction with lithium aluminumhydride, followed by oxidation by any of various methods used for oxidizing alcohols to aldehydes. It may be necessary to change the protecting group on the nitrogen during this sequence. The N-protected alkyl pyrrolidine-3-carboxylate, can, in turn, be accessed by azomethine cycloaddition to the corresponding acrylate ester.

Alternately the N-protected 3-formylpyrrolidine can be formed by treatment of N-protected 3-pyrrolidinone with methoxymethylenetriphenylphosphorane or other similar carbonyl homologation reactions known to those with skill in the art. The requisite N-protected 3-pyrrolidinone is made by sequential treatment of commercially available 3-pyrrolidinol with a suitable nitrogen-protecting agent and any of various oxidants used to convert alcohols to the corresponding ketones.

Alternatively, the N-protected 3-vinylpyrrolidines can be made from racemic or enantiomerically enriched N-protected 3-pyrrolidinol. One manner of accomplishing this transformation is to convert the hydroxyl group (of 3-pyrrolidinol) into the corresponding mesylate or tosylate and displacing the mesylate or tosylate with malonate ion. Subsequent hydrolysis of the malonate (to malonic acid), decarboxylation and lithium aluminumhydride reduction provides racemic or enantiomerically enriched (corresponding to the stereochemistry of the starting material) N-protected 3-(hydroxylethyl)pyrrolidine. This material can then be converted into the corresponding N-protected 3-(haloethyl)pyrrolidine, which can be dehydrohalogenated to give N-protected 3-vinylpyrrolidine (either racemic or enantiomerically enriched, corresponding to the stereochemistry of the starting material).

### III. Methods of Treatment

The compounds described herein are useful for treating those types of conditions and disorders for which other types of nicotinic compounds have been proposed as therapeutics. See, for example, Williams et al., DN&P 7(4):205-227 (1994), Arneric et al., CNS Drug Rev. 1(1):1-26 (1995), Arneric et al., Exp. Opin. Invest. Drugs 5(1):79-100 (1996), Bencherif et al., J. Pharmacol. Exp. Ther. 279:1413 (1996), Lippiello et al., J. Pharmacol. Exp. Ther. 279:1422 (1996), Damaj et al., Neuroscience (1997), Holladay et al., J. Med. Chem. 40(28): 4169-4194 (1997), Bannon et al., Science 279: 77-80 (1998), PCT WO 94/08992, PCT WO 96/31475, and U.S. Pat. Nos. 5,583,140 to Bencherif et al., 5,597,919 to Dull et al., and 5,604,231 to Smith et al.

The compounds can also be used as adjunct therapy in combination with existing therapies in the management of the aforementioned types of diseases and disorders. In such situations, it is preferably to administer the active ingredients in a manner that minimizes effects upon nAChR subtypes such as those that are associated with muscle and ganglia. This can be accomplished by targeted drug delivery and/or by adjusting the dosage such that a desired effect is obtained without meeting the threshold dosage required to achieve significant side effects. The pharmaceutical compositions can be used to ameliorate any of the symptoms associated with those conditions, diseases and disorders.

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, irritable bowel syndrome, Crohn's disease, ulcers, ulcerative colitis, acute cholangitis, aphthous stomatitis, cachexia, pouchitis, glomerulonephritis, lupus nephritis, thrombosis, and graft vs. host reaction.

One aspect of the present invention includes a method for relieving constipation. One embodiment of the present invention includes wherein the source of the constipation is: gastrointestinal, including but not limited to irritable bowel syndrome, including IBS-A and IBS-C, acute or chronic idiopathic constipation, colonic disorders including colon cancer, or ileus paralyticus; endrocrinological, including but not limited to pregnancy or hypothyroidism; neurological, including but mot limited to Parkinson's Disease, multiple schlerosis, or depression; iatrogenic, including but not limited to opiates, antidepressants, antacid medicines, or iron supplements; associated with an eating disorder, such as anorexia or bulimia as well as eating disorders associated with stress, travel, and dietary changes; or related to surgery, such as post-operative issues, injury, including spinal cord injury, autonomic dysfunction, or paraplegia, or long-term care patients, including oncology, CNS, stroke, paraplegic, and geriatric patients.

Another aspect of the present invention includes a method for treating constipation associated with a gastrointestinal disorder comprising administration of (R)-5-((E)-2-pyrrolidin-3-ylvinyl)pyrimidine or a pharmaceutically acceptable salt thereof. In one embodiment, the gastrointestinal disorder is irritable bowel syndrome, constipation predominant irritable bowel syndrome, alternating irritable bowel syndrome, chronic idiopathic constipation, acute constipation, drug-induced constipation, colonic disorders, colon cancer, or ileus paralyticus.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. Thus, when treating a CNS disorder, an effective amount of compound is an amount sufficient to pass across the blood-brain barrier of the subject, to bind to relevant receptor sites in the brain of the subject, and to activate relevant nicotinic receptor subtypes (e.g., provide neurotransmitter secretion, thus resulting in effective prevention or treatment of the disorder). Prevention of the disorder is manifested by delaying the onset of the symptoms of the disorder. Treatment of the disorder is manifested by a decrease in the symptoms associated with the disorder or an amelioration of the recurrence of the symptoms of the disorder.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to activate relevant receptors to affect neurotransmitter (e.g., dopamine) release but the amount should be insufficient to induce effects on skeletal muscles and ganglia to any significant degree. The effective dose of compounds will of course differ from patient to patient but in general includes amounts starting where CNS effects or other desired therapeutic effects occur, but below the amount where muscular effects are observed.

### IV. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising the compound of the present invention and one or more pharmaceutically acceptable carriers, diluents, or excipients. Another aspect of the invention provides a process for the preparation of a pharmaceutical composition including admixing the compound of the present invention with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

Pharmaceutical compositions may be formulated in unit dose form, or in multiple or subunit doses. The administration of the pharmaceutical compositions described herein can be intermittent, or at a gradual, continuous, constant, or controlled rate. In addition, the time of day and the number of times per day that the pharmaceutical composition is administered can vary.

The pharmaceutical compositions may be administered to any warm-blooded animal in need of relief of constipation, whether acute or chronic. For example, a mammal for such treatment includes a human being. Likewise, however, a mammal such as a mouse, rat, cat, rabbit, dog, pig, cow, horse, or monkey may be treated. With regard to the use of the present invention as either a human pharmaceutical or a veterinary product, the present invention may be used to relieve constipation from a variety of underlying causes.

The compound of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds.

In particular, the compound of the present invention may be used in conjunction with certain other therapeutic agents which are known to have constipation as a predominant side effect, including opioids, diuretics, antidepressants, antihistamines, antispasmodics, anticonvulsants, and aluminum antacids.

Likewise, the compound of the present invention may be used in conjunction with certain other therapeutic agents that are known or used for treatment of IBS, including but not limited to pain relievers, antibiotics, or secretagogues. In this regard, the present invention relieves a constipation portion of a disorder and, thus, may be combined with other agents for relief of other symptoms.

As further examples, the compound of the present invention can be used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

Enteric formulations of the present invention may include a core, either unitary or multi-particulate, and one or more coat. The one or more coat may be aesthetic or functional, including but not limited to pH-dependent and pH-independent functionality.

### A. Core

A particular multi-particulate core for a pellet is typically prepared by applying an active ingredient-containing layer to an inert core. Such inert cores are conventionally used in pharmaceutical science, and are readily available. A particular core is one prepared from starch and sucrose, for use in confectionery as well as in pharmaceutical manufacturing. However, cores of any pharmaceutically acceptable excipient can be used, including, for example, microcrystalline cellulose, vegetable gums, waxes, and the like. The primary characteristic of the inert core is to be inert, with regard both to the active ingredient and the other excipients in the pellet and with regard to the subject who will ultimately ingest the pellet.

The size of the cores depends on the desired size of the pellet to be manufactured. In general, pellets can be as small as 0.1 mm, or as large as 4 mm. Particular cores are from about 0.5 to about 3.0 mm, in order to provide finished pellets in the size range of from about 1.0 to about 3.0 mm in diameter.

The cores can be of a reasonably narrow particle size distribution, in order to improve the uniformity of the various coatings to be added and the homogeneity of the final product. For example, the cores can be specified as being of particle size ranges such as from 18 to 20 U.S. mesh, from 20 to 25 U.S. mesh, from 25 to 30 U.S. mesh, or from 30 to 35 U.S. mesh to obtain acceptable size distributions of various absolute sizes.

The amount of cores to be used can vary according to the weights and thicknesses of the added layers. In general, the cores comprise from about 10 to about 70 percent of the product. More particularly, the charge of cores represents from about 15 to about 45 percent of the product.

When manufacture of the pellet begins with inert cores, the active ingredient can be coated on the cores to yield a final drug concentration of about 10 to about 25 percent of the product, in general. The amount of active ingredient depends on the desired dose of the drug and the quantity of pellets to be administered. The dose of active ingredient is in the range of about 0.5-100 mg, more particularly about 1-10 mg, and the usual amount of pellets is that amount which is conveniently held in gelatin capsules. The volume of gelatin capsules can range of from about 15% to about 25% of active in the present product.

A convenient manner of coating the cores with active ingredient is the “powder coating” process where the cores are moistened with a sticky liquid or binder, active ingredient is added as a powder, and the mixture is dried. Such a process is regularly carried out in the practice of industrial pharmacy, and suitable equipment is known in the art.

Such equipment can be used in several steps of the present process. This process can be conducted in conventional coating pans similar to those employed in sugar coating processes. This process can be used to prepare pellets.

Alternately, the present product can be made in fluidized bed equipment (using a rotary processor), or in rotating plate equipment such as the Freund CF-Granulator (Vector Corporation, Marion, Iowa). The rotating plate equipment typically consists of a cylinder, the bottom of which is a rotatable plate. Motion of the mass of particles to be coated is provided by friction of the mass between the stationary wall of the cylinder and the rotating bottom. Warm air can be applied to dry the mass, and liquids can be sprayed on the mass and balanced against the drying rate as in the fluidized bed case.

In some embodiments, a powder coating is applied. In such embodiments, the mass of pellets can be maintained in a sticky state, and the powder to be adhered to them, active ingredient in this case, can be added continuously or periodically and adhered to the sticky pellets. When all of such active has been applied, the spray can be stopped and the mass allowed to dry in the air stream. It can be appropriate or convenient to add some inert powders to the active ingredient.

Additional solids can be added to the layer with active ingredient. These solids can be added to facilitate the coating process as needed to aid flow, reduce static charge, aid bulk buildup and form a smooth surface. Inert substances such as talc, kaolin, and titanium dioxide, lubricants such as magnesium stearate, finely divided silicon dioxide, crospovidone, and non-reducing sugars, e.g., sucrose, can be used. The amounts of such substances are in the range from about a few tenths of 1% of the product up to about 20% of the product. Such solids are typically of fine particle size, e.g., less than 50 micrometers, to produce a smooth surface.

The active ingredient can be made to adhere to the cores by spraying a pharmaceutical excipient which is sticky and adherent when it is wet, and dries to a strong, coherent film. Those skilled in the art are aware of and conventionally use many such substances, most of them polymers. Particular such polymers include hydroxypropylmethylcellulose, hydroxypropylcellulose and polyvinylpyrrolidone. Additional such substances include methylcellulose, carboxymethylcellulose, acacia and gelatin, for example. The amount of the adhering excipient can be in the range from about 4% to about 12% of the product, and depends in large part on the amount of active to be adhered to the core.

The active ingredient can also be built up on the cores by spraying a slurry comprising active suspended in a solution of the excipients of the active layer, dissolved or suspended in sufficient water to make the slurry sprayable. Such a slurry can be milled through a machine adapted for grinding suspension in order to reduce the particle size of active. Grinding in suspension form can be desirable because it avoids dust generation and containment problems which arise in grinding dry powder drugs. A particular method for applying this suspension is the pharmaceutical fluidized bed coating device, such as the Wurster column, which consists of a vertical cylinder with an air-permeable bottom and an upward spraying nozzle close above the bottom, or a downward-spraying nozzle mounted above the product mass. The cylinder is charged with particles to be coated, a sufficient volume of air is drawn through the bottom of the cylinder to suspend the mass of particles, and the liquid to be applied is sprayed onto the mass. The temperature of the fluidizing air is balanced against the spray rate to maintain the mass of pellets or tablets at the desired level of moisture and stickiness while the coating is built up.

On the other hand, the core can comprise a monolithic particle in which the active ingredient is incorporated. Such cores can be prepared by the granulation techniques which are wide spread in pharmaceutical science, particularly in the preparation of granular material for compressed tablets. The cores can be prepared by mixing the active into a mass of pharmaceutical excipients, moistening the mass with water or a solvent, drying, and breaking the mass into sized particles in the same size range as described above for the inert cores. This can be accomplished via the process of extrusion and marumerization.

The core for the pellet can also be prepared by mixing active with conventional pharmaceutical ingredients to obtain the desired concentration and forming the mixture into unitary cores of the desired size by conventional procedures, including but not limited to the process of R. E. Sparks et al., U.S. Pat. Nos. 5,019,302 and 5,100,592, incorporated by reference herein with regard to such process.

A particular protected core of the enteric pharmaceutical product comprises (R)-5-((E)-2-pyrrolidin-3-ylvinyl)pyrimidine (also referred to herein as Compound A) of the following formula (I):

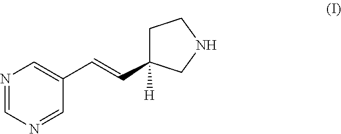 [SMILES: C(=C\[C@H]1CCNC1)/c1cncnc1]

as an active ingredient.

Methods for preparation of (R)-5-((E)-2-pyrrolidin-3-ylvinyl)pyrimidine are known in the art, as exemplified in U.S. Pat. No. 7,098,331, which is incorporated by reference herein in its entirety.

Also provided herein are oral compositions such as tablets or capsules containing said active ingredient which have a low excipient load such that once or twice a day dosing is possible, preferably with one or two such compositions being administered at each dosing. The enteric product provided herein can utilize any physical form of the active ingredient.

### B. Separating Layer

The separating layer between the active-containing core and the enteric layer is not required, but is a particular feature of the formulation. The functions of the separating layer, if desired, are to provide a smooth base for the application of the enteric layer, to prolong the resistance of the pellet to acid conditions, and/or to improve stability by inhibiting any interaction between the drug and the enteric polymer in the enteric layer.

The smoothing function of the separating layer is purely mechanical, the objective of which is to improve the coverage of the enteric layer and to avoid thin spots in it, caused by bumps and irregularities on the core. Accordingly, the more smooth and free of irregularities the core can be made, the less material is needed in the separating layer, and the need for the smoothing characteristic of the separating layer can be avoided entirely when the active is of extremely fine particle size and the core is made as close as possible to truly spherical.

When a pharmaceutically acceptable non-reducing sugar is added to the separating layer, the pellet's resistance to acid conditions can be markedly increased. Accordingly, such a sugar can be included in the separating layer applied to the cores, either as a powdered mixture, or dissolved as part of the sprayed-on liquid. A sugar-containing separating layer can reduce the quantity of enteric polymer required to obtain a given level of acid resistance. Use of less enteric polymer can reduce both the materials cost and processing time, and also can reduce the amount of polymer available to react with active. The inhibition of any core/enteric layer interaction is mechanical. The separating layer physically keeps the components in the core and enteric layers from coming into direct contact with each other. In some cases, the separating layer can also act as a diffusional barrier to migrating core or enteric layer components dissolved in product moisture. The separating layer can also be used as a light barrier by opacifying it with agents such as titanium dioxide, iron oxides and the like.

In general, the separating layer can include coherent or polymeric materials, and finely powdered solid excipients which constitute fillers. When a sugar is used in the separating layer, it is applied in the form of an aqueous solution and constitutes part of or the whole of the coherent material which sticks the separating layer together. In addition to or instead of the sugar, a polymeric material can also be used in the separating layer. For example, substances such as hydroxypropylmethylcellulose, polyvinylpyrrolidone, hydroxypropylcellulose and the like can be used in small amounts to increase the adherence and coherence of the separating layer.

A filler excipient also can be used in the separating layer to increase the smoothness and solidity of the layer. Substances such as finely powered talc, silicon dioxide and the like are universally accepted as pharmaceutical excipients and can be added as is convenient in the circumstances to fill and smooth the separating layer.

In general, the amount of sugar in the separating layer can be in the range of from about 2% to about 10% of the product, when a sugar is used at all, and the amount of polymeric or other sticky material can be in the range of from about 0.1 to about 5%. The amount of filler, such as talc, can be in the range of from about 5 to about 15%, based on final product weight.

The separating layer can be applied by spraying aqueous solutions of the sugar or polymeric material, and dusting in the filler as has been described in the preparation of an active layer. The smoothness and homogeneity of the separating layer can be improved, however, if the filler is thoroughly dispersed as a suspension in the solution of sugar and or polymeric material, and the suspension is sprayed on the core and dried, using equipment as described above in the preparation of cores with active layers.

### C. Enteric Layer

The enteric layer is comprised of an enteric polymer, which can be chosen for compatibility with the active ingredient. The polymer can be one having only a small number of carboxylic acid groups per unit weight or repeating unit of the polymer.

In general, the release rate for the active pharmaceutical agent (whether hydrophilic, hydrophobic or amphiphilic) can be controlled by adjusting the thickness and/or composition of the coating, and, optionally, by adjusting the type and/or concentration of the polymeric and/or non-polymeric excipients.

The release rate is suppressed with the polymer in the core, because the molecular weight of the polyethylene oxide is relatively high. An additional advantage of using relatively high molecular weight polyethylene oxide is that the release is pH independent, unlike where ionic polymers such as polyacrylic acids are used. Further, active pharmaceutical agents including functional groups that might react with such polymers (i.e., that include amine and/or carboxylic acid groups) can be used without an adverse reaction between the active agent and the polymer.

Enteric polymers can be applied as coatings from aqueous suspensions, from solutions in aqueous or organic solvents, or as a powder. One skilled in the art will be able to select from known solvents and/or methods as desired.

A particular enteric polymer is an acrylic drug delivery polymer, such as polymethacrylates, such as those sold under the tradename Eudragit®, including powder applications such as Eudragit® L100-55 and L100.

The enteric polymer can also be applied according to a method described by Shin-Etsu Chemical Co. Ltd. (Obara, et al., Poster PT6115, AAPS Annual Meeting, Seattle, Wash., Oct. 27-31, 1996). In this method, when the enteric polymer is applied as a powder the enteric polymer is added directly in the solid state to the tablets or pellets while plasticizer is sprayed onto the tablets or pellets simultaneously. The deposit of solid enteric particles is then turned into a film by curing. The curing is done by spraying the coated tablets or pellets with a small amount of water and then heating the tablets or pellets for a short time. This method of enteric coating application can be performed employing the same type of equipment as described above in the preparation of cores with active ingredient layers.

When the enteric polymer is applied as an aqueous suspension, a problem in obtaining a uniform, coherent film often results. In instances in which this problem may arise, a fine particle grade can be used or the particles of polymer can be ground to an extremely small size before application. It is possible either to grind the dry polymer, as in an air-impaction mill or to prepare the suspension and grind the polymer in slurry form. Slurry grinding is generally preferable, particularly since it can be used also to grind the filler portion of the enteric layer in the same step. In some embodiments, it is advisable to reduce the average particle size of the enteric polymer to the range from about 1 micrometer to about 5 micrometers, particularly no larger than 3 micrometers.

When the enteric polymer is applied in the form of a suspension, the suspension is typically maintained homogeneous. Such precautions include maintaining the suspension in a gently stirred condition, but not stirring so vigorously as to create foam, and assuring that the suspension does not stand still in eddies in nozzle bodies, for example, or in over-large delivery tubing. Frequently, polymers in suspension form will agglomerate if the suspension becomes too warm, and the critical temperature can be as low as 30° C. in individual cases. Since spray nozzles and tubing are exposed to hot air in the usual fluid bed type equipment, care must be taken to assure that the suspension is kept moving briskly through the equipment to cool the tubing and nozzle. When HPMCAS is used, in particular, it is advisable to cool the suspension below 20° C. before application, to cool the tubing and nozzle by pumping a little cold water through them before beginning to pump the suspension, and to use supply tubing with as small a diameter as the spray rate will allow so that the suspension can be kept moving rapidly in the tubing.

In one embodiment, one can apply the enteric polymer as an aqueous solution whenever it is possible to do so. Dissolution of the polymer can be obtained by neutralizing the polymer, particularly with ammonia. Neutralization of the polymer can be obtained merely by adding ammonia, preferably in the form of aqueous ammonium hydroxide to a suspension of the polymer in water; complete neutralization results in complete dissolution of the polymer at about pH 5.7-5.9. Good results are also obtained when the polymer is partially neutralized by adding less than the equivalent amount of ammonia. In such case, the polymer which has not been neutralized remains in suspended form, suspended in a solution of neutralized polymer. The particle size of the polymer can be controlled when such a process is to be used. Use of neutralized polymer more readily provides a smooth, coherent enteric layer than when a suspended polymer is used, and use of partially neutralized polymer provides intermediate degrees of smoothness and coherency. Particularly when the enteric layer is applied over a very smooth separating layer, excellent results can be obtained from partially neutralized enteric polymer.

The extent of neutralization can be varied over a range without adversely affecting results or ease of operation. For example, the extent of neutralization can range from about 25% to about 100% neutralization. Another particular condition is from about 45% to about 100% neutralization, and another condition is from about 65% to about 100%. Still another particular manner of neutralization is from about 25% to about 65% neutralized. It may be found, however, that the enteric polymer in the resulting product, after drying, is neutralized to a lesser extent than when applied.

A plasticizer can be used with enteric polymers for improved results. A particular plasticizer can be triethyl citrate, used in an amount up to about 15%-30% of the amount of enteric polymer in aqueous suspension application. Either lower levels or no plasticizer can be required. Minor ingredients, such as antifoam, suspending agents (when the polymer is in suspended form), or surfactants to assist in smoothing the film, are also commonly used. For example, silicone anti-foams, surfactants such as polysorbate 80, sodium lauryl sulfate and the like and suspending agents such as carboxymethylcellulose, vegetable gums and the like, can commonly be used at amounts in the general range up to 1% of the product.

An enteric layer may be filled with a powdered excipient such as talc, glyceryl monostearate or hydrated silicon dioxide to build up the thickness of the layer, to strengthen it, to reduce static charge, and to reduce particle cohesion. Amounts of such solids in the range of from about 1% to about 10% of the final product can be added to the enteric polymer mixture, while the amount of enteric polymer itself can be in the range from about 5% to about 25%, more particularly, from about 10% to about 20%.

Application of the enteric layer to the pellets follows the same general procedure previously discussed, using fluid bed type equipment with simultaneous spraying of enteric polymer solution or suspension and warm air drying. Temperature of the drying air and the temperature of the circulating mass of pellets are typically kept in the ranges advised by the manufacturer of the enteric polymer.

### D. Finishing Layer

A finishing layer over the enteric layer is not necessary in every case, but can improve the elegance of the product and its handling, storage and machinability and can provide further benefits as well. The simplest finishing layer is simply a small amount, about less than 1% of an anti-static ingredient such as talc or silicon dioxide, simply dusted on the surface of the pellets. Another simple finishing layer is a small amount, about 1%, of a wax such as beeswax melted onto the circulating mass of pellets to further smooth the pellets, reduce static charge, prevent any tendency for pellets to stick together, and increase the hydrophobicity of the surface.

More complex finishing layers can constitute a final sprayed-on layer of ingredients. For example, a thin layer of polymeric material such as hydroxypropylmethylcellulose, polyvinylpyrrolidone and the like, in an amount such as from about 2% up to about 10%, can be applied. The polymeric material can also carry a suspension of an opacifier, a bulking agent such as talc, or a coloring material, particularly an opaque finely divided color agent such as red or yellow iron oxide. Such a layer quickly dissolves away in the stomach, leaving the enteric layer to protect the active ingredient, but provides an added measure of pharmaceutical elegance and protection from mechanical damage to the product.

Finishing layers to be applied to the present product are of essentially the same types commonly used in pharmaceutical science to smooth, seal and color enteric products, and can be formulated and applied in the usual manners.

Pellets made according to the above examples, and gelatin capsules filled with various batches of such pellets, are thoroughly tested in the manners usual in pharmaceutical science. Results of stability tests show that the pellets and capsules have sufficient storage stability to be distributed, marketed and used in the conventional pharmaceutical manner.

The pellets and capsules are believed to pass the conventional tests for enteric protection under conditions prevailing in the stomach. Pellets are believed to release their load of drug product acceptably quickly when exposed to conditions prevailing in the small intestine.

The enteric coated particles may be filled into HDP #1 capsules. The dissolution profile of the capsules batches in HCl 0.1 N, based on USP procedures, may be taken. The dissolution profile of the capsules in phosphate buffer may be measured. The dissolution profile is expected to show that the enteric coating is effective in protecting the spheres from being dissolved in the stomach, and are easily soluble in intestine-like conditions.

The present invention includes tables and capsules in quantities of active ingredient ranging from, for example, 0.5-50 mg, including 1-10 mg, further including 5 mg.

### Example 1

### Racemic 5-((E)-2-pyrrolidin-3-ylvinyl)pyrimidine hemigalactarate

Trifluoroacetic acid (1.2 cm³, 15.6 mmol) was added drop-wise to a solution of 0.43 g (1.56 mmol) of racemic 3-((E)-2-pyrimidin-5-ylvinyl)pyrrolidine-1-carboxylic acid tert-butyl ester in 6 cm³ of dichloromethane, which was under argon and cooled to 0° C. The reaction mixture was stirred at this temperature for 0.5 h then at a temperature in the region of 22° C. for 20 hours and it was concentrated to dryness under reduced pressure (2.7 kPa). The oily residue was taken up in 5 cm³ of water and the resulting solution was rendered basic (pH=8) by adding 28% aqueous ammonia solution and was then extracted with 3 times 25 cm³ of dichloromethane. The combined organic phases were washed with 25 cm³ of water, dried over magnesium sulfate, filtered and concentrated to dryness under reduced pressure (2.7 kPa) to give 0.126 g of orange-colored oil which was purified by chromatography on silica gel [eluent: dichloromethane/methanol (9/1 then 8/2 by volume)]. Concentration of the fractions under reduced pressure (2.7 kPa) gave 0.1 g (0.57 mmol) of orange-colored oil. Galactaric acid (0.06 g, 0.28 mmol) was added to a solution of this oil in 2 cm³ of methanol to which 0.5 cm³ of water has been added. The mixture was brought to reflux and cooled to a temperature in the region of 22° C. and the insoluble material was removed by filtration. The filtrate was concentrated to dryness under reduced pressure (2.7 kPa) and the oily residue was taken up in 2 cm³ of ethanol. The precipitated solid was filtered off, washed with 2 cm³ of isopropyl acetate and 2 cm³ of diisopropyl ether and then dried at 40° C. under vacuum (2.7 kPa) to give 0.1 g of racemic 5-((E)-2-pyrrolidin-3-ylvinyl)pyrimidine hemigalactarate in the form of an ochre solid. Mass spectrum (DCI): m/z 176 (MH⁺). ¹H NMR spectrum (300 MHz, (CD₃)₂SO d6 with a few drops of CD₃COOD d4, δ in ppm): 1.82 (m: 1H); 2.18 (m: 1H); 2.98 (dd, J=11 and 8.5 Hz: 1H); 3.10 (m: 1H); 3.20 (m: 1H); 3.33 (m: 1H); 3.42 (dd, J=11 and 7.5 Hz: 1H); 3.79 (s: 1H); 4.24 (s: 1H); 6.55 (limit AB: 2H); 8.87 (s: 2H); 9.04 (s: 1H).

Racemic 3-((E)-2-pyrimidin-5-ylvinyl)pyrrolidine-1-carboxylic acid tert-butyl ester can be prepared as follows:

Palladium acetate (0.117 g, 0.52 mmol), 0.678 g (16 mmol) of lithium chloride and 7.25 cm³ (42 mmol) of ethyldiisopropylamine were added in succession to a solution under argon of 0.822 g (5.17 mmol) of 5-bromopyrimidine and 1.2 g (5.17 mmol) of racemic 3-vinylpyrrolidine-1-carboxylic acid tert-butyl ester in 15 cm³ of dimethylformamide. After 3 hours of heating at 110° C. with stirring, the reaction mixture was stirred for 2 hours at a temperature in the region of 22° C. and then concentrated to dryness under reduced pressure (2.7 kPa). The oily residue was taken up in 50 cm³ of ethyl acetate and the resulting solution was washed in succession with 2 times 25 cm³ of water, 25 cm³ of saturated bicarbonate solution, 2 times 25 cm³ of water and 25 cm³ of saturated sodium chloride solution and was then dried over magnesium sulfate, filtered and concentrated to dryness under reduced pressure (2.7 kPa) to give 1.1 g of brown oil. This residue was purified by chromatography on silica gel [eluent: cyclohexane/ethyl acetate (8/2 by volume)]. Concentration of the fractions under reduced pressure (2.7 kPa) gave 0.43 g of racemic 3-((E)-2-pyrimidin-5-ylvinyl)pyrrolidine-1-carboxylic acid tert-butyl ester in the form of an oil.

¹H NMR spectrum (300 MHz, (CD₃)₂SO d6, δ in ppm): 1.42 (s: 9H); 1.78 (m: 1H); 2.05 (m: 1H); from 2.90 to 3.15 (m: 2H); from 3.15 to 3.60 (m: 3H); 6.51 (d, J=16.5 Hz: 1H); 6.64 (dd, J=16.5 and 7 Hz: 1H); 8.89 (s: 2H); 9.04 (s: 1H).

### Example 2

### (S)-5-((E)-2-Pyrrolidin-3-ylvinyl)pyrimidine galactarate

Trimethylsilyl iodide (0.2 cm³, 1.4 mmol) was added at a temperature in the region of 22° C. to a solution under argon of 0.26 g (0.944 mmol) of (S)-3-((E)-2-pyrimidin-5-ylvinyl)pyrrolidine-1-carboxylic acid tert-butyl ester in 10 cm³ of dichloromethane. After 2 hours of stirring at this temperature the reaction mixture was admixed with 15 cm³ of 5% aqueous ammonia solution and stirred for 1 hour at a temperature in the region of 22° C. and left to settle. The aqueous phase was separated and extracted with dichloromethane. The combined organic phases were washed twice with water and with saturated aqueous sodium chloride solution and were then dried over magnesium sulfate, filtered and concentrated to dryness under reduced pressure (2.7 kPa) to give 0.06 g of orange-colored oil. Galactaric acid (0.035 g, 0.16 mmol) was added to a solution of this oil in 6 cm³ of methanol to which 0.6 cm³ of water has been added. The mixture was brought to reflux, cooled to a temperature in the region of 22° C. and concentrated to dryness under reduced pressure (2.7 kPa). The oily residue was triturated in the presence of 5 cm³ of diisopropyl ether and the solid formed was filtered off and then dried at 45° C. under vacuum (2.7 kPa) to give 0.072 g of (S)-5-((E)-2-pyrrolidin-3-ylvinyl)pyrimidine galactarate in the form of a yellow solid. Mass spectrum (DCI): m/z=176 (MH⁺). ¹H NMR spectrum (300 MHz, (CD₃)₂SO d6 with a few drops of CD₃COOD d4, δ in ppm): 1.81 (m: 1H); 2.19 (m: 1H); 2.98 (dd, J=11 and 9 Hz: 1H); 3.10 (m: 1H); 3.21 (m: 1H); 3.33 (m: 1H); 3.43 (dd, J=11 and 8 Hz: 1H); 3.79 (s: 2H); 4.25 (s: 2H); 6.56 (limit AB: 2H); 8.88 (s: 2H); 9.05 (s: 1H).

(S)-3-((E)-2-Pyrimidin-5-ylvinyl)pyrrolidine-1-carboxylic acid tert-butyl ester can be prepared as follows:

A racemic mixture of 3-((E)-2-pyrimidin-5-ylvinyl)pyrrolidine-1-carboxylic acid tert-butyl ester (0.5 g) was injected in two parts on a 8 cm diameter column containing 1.2 kg of chiral stationary phase Chiralpak AS™ 20 μm [flow: 130 ml/min, eluent: heptane/methanol/ethanol (98/1/1 by volume)]. Concentration of the fractions under reduced pressure (2.7 kPa) gave 0.24 g of (S)-((E)-2-Pyrimidin-5-ylvinyl)pyrrolidine-1-carboxylic acid tert-butyl ester and 0.27 g of (R)-((E)-2-Pyrimidin-5-ylvinyl)pyrrolidine-1-carboxylic acid tert-butyl ester. (+)-((E)-2-Pyrimidin-5-ylvinyl)pyrrolidine-1-carboxylic acid tert-butyl ester was eluted in first position with a retention time of 14.2 min on a 4.6 mm diameter and 250 mm length Chiralpak AS™ 20 μm column [flow: 1 ml/min, eluent: heptane/methanol/ethanol (98/1/1 by volume)].

¹H NMR spectrum (300 MHz, (CD₃)₂SO d6, δ in ppm): 1.43 (s: 9H); 1.79 (m: 1H); 2.06 (m: 1H); from 2.95 to 3.15 (m: 2H); from 3.20 to 3.35 (m: 1H); 3.44 (ddd, J=11-8.5 and 3 Hz: 1H); 3.53 (broad dd, J=10 and 7.5 Hz: 1H); 6.52 (d, J=16.5 Hz: 1H); 6.63 (dd, J=16.5 and 7 Hz: 1H); 8.89 (s: 2H); 9.04 (s: 1H).

(R)-((E)-2-Pyrimidin-5-ylvinyl)pyrrolidine-1-carboxylic acid tert-butyl ester was eluted in second position with a retention time of 17 min on a 4.6 mm diameter and 250 mm length Chiralpak AS™ 20 μm column [flow: 1 ml/min, eluent: heptane/methanol/ethanol (98/1/1 by volume)]. ¹H NMR spectrum (300 MHz, (CD₃)₂SO d6, δ in ppm): 1.43 (s: 9H); 1.79 (m: 1H); 2.06 (m: 1H); from 2.95 to 3.15 (m: 2H); from 3.20 to 3.35 (m: 1H); 3.44 (ddd, J=11-8.5 and 3 Hz: 1H); 3.53 (broad dd, J=10 and 7.5 Hz: 1H); 6.52 (d, J=16.5 Hz: 1H); 6.63 (dd, J=16.5 and 7 Hz: 1H); 8.89 (s: 2H); 9.04 (s: 1H).

### Example 3

### (R)-5-((E)-2-pyrrolidin-3-ylvinyl)pyrimidine galactarate

Trimethylsilyl iodide (0.2 cm³, 1.4 mmol) was added at a temperature in the region of 22° C. to a solution under argon of 0.29 g (1.053 mmol) of (−)-3-((E)-2-pyrimidin-5-ylvinyl)pyrrolidine-1-carboxylic acid tert-butyl ester in 10 cm³ of dichloromethane. After 2 hours of stirring at this temperature the reaction mixture was admixed with 15 cm³ of 5% aqueous ammonia solution, stirred for 1 h at a temperature in the region of 22° C. and left to settle. The aqueous phase was separated off and extracted with dichloromethane. The combined organic phases were washed twice with water and with saturated aqueous sodium chloride solution and then were dried over magnesium sulfate, filtered and concentrated to dryness under reduced pressure (2.7 kPa) to give 0.1 g of orange-colored oil. Galactaric acid (0.06 g, 0.28 mmol) was added to a solution of this oil in 10 cm³ of methanol to which 1 cm³ of water has been added. The mixture was brought to reflux, cooled to a temperature in the region of 22° C. and concentrated to dryness under reduced pressure (2.7 kPa). The oily residue was triturated in the presence of 5 cm³ of diisopropyl ether and the solid formed was filtered and then dried at 45° C. under vacuum (2.7 kPa) to give 0.094 g of (R)-5-((E)-2-pyrrolidin-3-ylvinyl)pyrimidine galactarate in the form of a yellow solid. Mass spectrum (DCI): m/z=176 (MH⁺).

¹H NMR spectrum (300 MHz, (CD₃)₂SO d6 with a few drops of CD₃COOD d4, δ in ppm): 1.82 (m: 1H); 2.19 (m: 1H); 2.98 (dd, J=11 and 9 Hz: 1H); 3.10 (m: 1H); 3.21 (m: 1H); 3.32 (m: 1H); 3.43 (dd, J=11 and 7.5 Hz: 1H); 3.79 (s: 2H); 4.24 (s: 2H); 6.57 (limit AB: 2H); 8.88 (s: 2H); 9.05 (s: 1H).

(R)-3-((E)-2-pyrimidin-5-ylvinyl)pyrrolidine-1-carboxylic acid tert-butyl ester can be prepared as described in Example 2.

### Example 4

### Synthesis of tert-butyl (R)-3-(methylsulfonyloxy)pyrrolidine-1-carboxylate (2)

Procedure A:

To a solution of tert-butyl (R)-3-hydroxypyrrolidine-1-carboxylate (200 g, 1.07 mol) and triethylamine (167 g, 1.63 mol) in toluene (700 mL) at −20 to −30° C. was added methanesulfonyl chloride (156 g, 1.36 mol) drop-wise while maintaining the temperature at −10 to −20° C. The solution was warmed to ambient temperature and allowed to stir. The reaction solution was sampled hourly and analyzed by HPLC to establish completion of the reaction. Upon completion of the reaction, the suspension was filtered to remove the triethylamine hydrochloride. The filtrate was washed with ˜600 mL of dilute aqueous sodium bicarbonate solution. The organic layer was dried and concentrated under reduced pressure to give 2 as a viscous oil (260 g, 92%) which is used without further purification. ¹H NMR (CDCl₃, 400 MHz) δ 5.27 (m, 1H), 3.44-3.76 (m, 4H), 3.05 (s, 3H), 2.26 (m, 1H), 2.15 (m, 1H), 1.47 (s, 9H).

Procedure B:

A reactor was charged with tert-butyl (R)-3-hydroxypyrrolidine-1-carboxylate (2.00 kg, 10.7 mol), toluene (8.70 kg) and triethylamine (1.75 kg, 17.3 mol). The reactor was flushed with nitrogen for 15 min. The mixture was stirred and cooled to 3° C. Methanesulfonyl chloride (1.72 kg, mol) was slowly added (over a 2 h period) with continuous ice bath cooling (exothermic reaction) (after complete addition, the temperature was 14° C.). The mixture, now viscous with precipitated triethylamine hydrochloride, was stirred 12 h as it warmed to 20° C. Both GC and TLC analysis (ninhydrin stain) indicated that no starting material remained. The mixture was filtered to remove the triethylamine hydrochloride, and the filtrate was returned to the reactor. The filtrate was then washed (2×3 kg) with 5% aqueous sodium bicarbonate, using 15 min of stirring and 15 min of settling time for each wash. The resulting organic layer was dried over anhydrous sodium sulfate and filtered. The volatiles were removed from the filtrate under vacuum, first at 50° C. for 4 h and then at ambient temperature for 10 h. The residue weighed 3.00 kg (106% yield) and was identical by chromatographic and NMR analysis to previously prepared samples, with the exception that it contained toluene.

### Example 5

### Synthesis of diethyl (R)-2-(1-(tert-butoxycarbonyl)pyrrolidin-3-yl)malonate (3)

Preparation A:

To a solution of potassium tert-butoxide (187 g, 1.62 mol) in 1-methyl-2-pyrrolidinone (1.19 L) was added diethyl malonate (268 g. 1.67 mol) while maintaining the temperature below 35° C. The solution was heated to 40° C. and stirred for 20-30 min. tert-Butyl (R)-3-(methylsulfonyloxyl)pyrrolidine-1-carboxylate (112 g, 420 mmol) was added and the solution was heated to 65° C. and stirred for 6 h. The reaction solution was sampled every 2 h and analyzed by HPLC to establish completion of the reaction. Upon completion of reaction (10-12 h), the mixture was cooled to around 25° C. De-ionized water (250 mL) was added to the solution, and the pH was adjusted to 3-4 by addition of 2N hydrochloric acid (650 mL). The resulting suspension was filtered, and water (1.2 L) and chloroform (1.4 L) were added. The solution was mixed thoroughly, and the chloroform layer was collected and evaporated under reduced pressure to give a yellow oil. The oil was dissolved in hexanes (2.00 L) and washed with deionized water (2×1.00 L). The organic layer was concentrated under reduced pressure at 50-55° C. to give a pale yellow oil (252 g) which ¹H NMR analysis indicates to be 49.1% of 3 (123.8 g) along with 48.5% diethyl malonate (122 g), and 2% of 1-methyl-2-pyrrolidinone (5 g). The material was carried forward to the next step without further purification. ¹H NMR (CDCl₃, 400 MHz) δ 4.20 (q, 4H), 3.63 (m, 1H), 3.48 (m, 1H), 3.30 (m, 1H), 3.27 (d, J=10 Hz, 1H), 3.03 (m, 1H), 2.80 (m, 1H), 2.08 (m, 1H), 1.61 (m, 1H), 1.45 (s, 9H), 1.27 (t, 6H).

Preparation B:

A reactor, maintained under a nitrogen atmosphere, was charged with 200 proof ethanol (5.50 kg) and 21% (by weight) sodium ethoxide in ethanol (7.00 kg, 21.6 mol). The mixture was stirred and warmed to 30° C. Diethyl malonate (3.50 kg, 21.9 mol) was added over a 20 min period. The reaction mixture was then warmed at 40° C. for 1.5 h. A solution of tert-butyl (R)-3-(methylsulfonyloxyl)pyrrolidine-1-carboxylate (3.00 kg of the product from Example 2, Procedure B, 10.7 mol) in 200 proof ethanol (5.50 kg) was added, and the resulting mixture was heated at reflux (78° C.) for 2 h. Both GC and TLC analysis (ninhydrin stain) indicated that no starting material remained. The stirred mixture was then cooled to 25° C., diluted with water (2.25 kg), and treated slowly with a solution of concentrated hydrochloric acid (1.27 kg, 12.9 mol) in water (5.44 kg). This mixture was washed twice with methyl tert-butyl ether (MTBE) (14.1 kg and 11.4 kg), using 15 min of stirring and 15 min of settling time for each wash. The combined MTBE washes were dried over anhydrous sodium sulfate (1 kg), filtered and concentrated under vacuum at 50° C. for 6 h. The residue (red oil) weighed 4.45 kg and was 49% desired product by GC analysis (62% overall yield from tert-butyl (R)-3-hydroxypyrrolidine-1-carboxylate).

### Example 6

### Synthesis of (R)-2-(1-(tert-butoxycarbonyl)pyrrolidin-3-yl)malonic acid (4)

Procedure A:

To a solution of the product of Example 3, Procedure A (232 g), containing 123.8 g (380 mmol) of 3 and 121.8 g (760 mmol) of diethyl malonate, in tetrahydrofuran (1.2 L) was added a 21% potassium hydroxide solution (450 g in 0.50 L of deionized water) while maintaining the temperature below 25° C. The reaction mixture was heated to 45° C. and stirred for 1 h. The reaction solution was sampled every hour and analyzed by HPLC to establish completion of the reaction. Upon completion of reaction (2-3 h), the mixture was cooled to around 25° C. The aqueous layer was collected and cooled to 5° C. The pH was adjusted to 2 by addition of 4N hydrochloric acid (750 mL), and the resulting suspension was held at 5-10° C. for 30 min. The mixture was filtered, and the filter cake was washed with hexanes (1 L). The aqueous filtrate was extracted with chloroform (1 L) and the chloroform layer was put aside. The solids collected in the filtration step were re-dissolved in chloroform (1 L) by heating to 40° C. The solution was filtered to remove un-dissolved inorganic solids. The chloroform layers were combined and concentrated under reduced pressure at 50-55° C. to give an off-white solid (15 g). The solids were combined and dissolved in ethyl acetate (350 mL) to give a suspension that was warmed to 55-60° C. for 2 h. The suspension was filtered while hot and the resulting cake washed with ethyl acetate (2×150 mL) and hexanes (2×250 mL) to give 83.0 g (80.1%) of 4 as a white solid which was used in the next step without further purification. ¹H NMR (d₄-CH₃OH, 400 MHz) δ 3.60 (m, 1H), 3.46 (m, 1H), 3.29-3.32 (m, 2H), 2.72 (m, 1H), 2.09 (m, 1H), 1.70 (m, 1H), 1.45 (s, 9H).

Procedure B:

A solution of the product of Example 3, Procedure B (4.35 kg), containing 2.13 kg (6.47 mol) of 3, in tetrahydrofuran (13.9 kg) was added to a stirred, cooled solution of potassium hydroxide (1.60 kg, 40.0 mol) in deionized water (2.00 kg) under a nitrogen atmosphere, while maintaining the temperature below 35° C. The reaction mixture was heated and maintained at 40-45° C. for 24 h, by which time GC and TLC analysis indicated that the reaction was complete. The mixture was cooled to 25° C. and washed with MTBE (34 kg), using 15 min of stirring and 15 min of settling time. The aqueous layer was collected and cooled to 1° C. A mixture of concentrated hydrochloric acid (2.61 kg, 26.5 mol) in deionized water (2.18 kg) was then added slowly, keeping the temperature of the mixture at <15° C. during and for 15 min after the addition. The pH of the solution was adjusted to 3.7 by further addition of hydrochloric acid. The white solid was collected by filtration, washed with water (16 kg), and vacuum dried at ambient temperature for 6 d. The dry solid weighed 1.04 kg. The filtrate was cooled to <10° C. and kept at that temperature as the pH was lowered by addition of more hydrochloric acid (1.6 L of 6 N was used; 9.6 mol; final pH=2). The white solid was collected by filtration, washed with water (8 L), and vacuum dried at 40° C. for 3 d. The dry solid weighed 0.25 kg. The combined solids (1.29 kg, 73% yield) were chromatographically identical to previously prepared samples.

### Example 7

### Synthesis of (R)-2-(1-(tert-butoxycarbonyl)pyrrolidine-3-yl)acetic acid (5)

Procedure A:

A solution of (R)-2-(1-(tert-butoxycarbonyl)pyrrolidin-3-yl)malonic acid (83 g) in 1-methyl-2-pyrrolidinone (0.42 L) was stirred under nitrogen at 110-112° C. for 2 h. The reaction solution was sampled every hour and analyzed by HPLC to establish completion of the reaction. Upon completion of reaction the reaction solution was cooled to 20-25° C. The solution was mixed with de-ionized water (1.00 L), and MTBE (1.00 L) was added. The phases were separated, and the organic layer was collected. The aqueous phase was extracted with MTBE (1.00 L), then chloroform (1.00 L). The organic layers were combined and concentrated under reduced pressure at 50-55° C. to give an oil. This oil was dissolved in MTBE (2.00 L) and washed twice with 0.6N hydrochloric acid (2×1.00 L). The organic layer was collected and concentrated under reduced pressure at 50-55° C. to give a semi-solid. The semi-solid was suspended in 1:4 ethyl acetate/hexanes (100 mL), heated to 50° C., held for 30 min, cooled to −10° C., and filtered. The filtrate was concentrated under reduced pressure to give an oil, which was dissolved in MTBE (250 mL) and washed twice with 0.6N hydrochloric acid (2×100 mL). The organic layer was concentrated under reduced pressure at 50-55° C. to give a semi-solid which was suspended in 1:4 ethyl acetate/hexanes (50 mL), heated to 50° C., held for 30 min, cooled to −10° C., and filtered. The solids were collected, suspended in hexanes (200 mL), and collected by filtration to give 54.0 g (77.6%) of 5. ¹H NMR (CDCl₃, 400 MHz) δ 11.00 (br s, 1H), 3.63 (m, 1H), 3.45 (M, 1H), 3.30 (M, 1H), 2.97 (m, 1H), 2.58 (m, 1H), 2.44 (m, 2H), 2.09 (m, 1H), 1.59 (M, 1H), 1.46 (s, 9H).

Procedure B:

A solution of (R)-2-(1-(tert-butoxycarbonyl)pyrrolidin-3-yl)malonic acid (1.04 kg, 3.81 mol) in 1-methyl-2-pyrrolidinone (6.49 kg) was stirred under nitrogen at 110° C. for 5 h, by which time TLC and HPLC analysis indicated that the reaction was complete. The reaction mixture was cooled to 25° C. (4 h) and combined with water (12.8 kg) and MTBE (9.44 kg). The mixture was stirred vigorously for 20 min, and the phases were allowed to separate (10 h). The organic phase was collected, and the aqueous phase was combined with MTBE (9.44 kg), stirred for 15 min, and allowed to settle (45 min). The organic phase was collected, and the aqueous phase was combined with MTBE (9.44 kg), stirred for 15 min, and allowed to settle (15 min). The three organic phases were combined and washed three times with 1N hydrochloric acid (8.44 kg portions) and once with water (6.39 kg), using 15 min of stirring and 15 min of settling time for each wash. The resulting solution was dried over anhydrous sodium sulfate (2.0 kg) and filtered. The filtrate was concentrated under reduced pressure at 31° C. (2 h) to give an solid. This solid was heated under vacuum for 4 h at 39° C. for 4 h and for 16 h at 25° C., leaving 704 g (81%) of 5 (99.7% purity by GC).

Procedure C (Streamlined Synthesis of 5, Using 2 as Starting Material):

A stirred mixture of sodium ethoxide in ethanol (21 weight percent, 343 g, 1.05 mol), ethanol (anhydrous, 300 mL) and diethyl malonate (168 g, 1.05 mol) was heated to 40° C. for 1.5 h. To this mixture was added a solution of (R)-tert-butyl 3-(methylsulfonyloxy)pyrrolidine-1-carboxylate (138 g, 0.592 mol) in ethanol (100 mL) and the reaction mixture was heated to 78° C. for 8 h. The cooled reaction mixture was diluted with water (2.0 L) and acidified to pH=3 with 6M HCl (100 mL). The aqueous ethanol mixture was extracted with toluene (1.0 L), and the organic phase concentrated under vacuum to afford 230 g of a red oil. The red oil was added at 85° C. to a 22.5 weight percent aqueous potassium hydroxide (748 g, 3.01 mol). After the addition was complete, the reaction temperature was allowed to slowly rise to 102° C. while a distillation of ethanol ensued. When the reaction temperature had reached 102° C., and distillation had subsided, heating was continued for an additional 90 min. The reaction mixture was cooled to ambient temperature and washed with toluene (2×400 mL). To the aqueous layer was added 600 mL 6M hydrochloric acid, while keeping the internal temperature below 20° C. This resulted in the formation of a precipitate, starting at pH of about 4-5. The suspension was filtered, and the filter cake was washed with 300 mL water. The solid was dried under vacuum to afford 77 g of (R)-2-(1-(tert-butoxycarbonyl)pyrrolidin-3-yl)malonic acid as an off-white solid (54% yield with respect to (R)-tert-butyl 3-(methylsulfonyloxy)pyrrolidine-1-carboxylate). ¹H NMR (DMSO-d₆, 400 MHz): δ 3.47 (m, 1H); 3.32 (m, 1H); 3.24 (m, 1H); 3.16 (m, 1H); 3.92 (m, 1H); 2.86 (m, 1H); 1.95 (m, 1H); 1.59 (m, 1H); 1.39 (s, 9H).

A suspension of (R)-2-(1-(tert-butoxycarbonyl)pyrrolidin-3-yl)malonic acid (15 g, 55 mmol) in toluene (150 mL) and dimethylsulfoxide (2 mL) was heated to reflux for a period of 2 h. The mixture was allowed to reach ambient and diluted with MTBE (150 mL). The organic solution was washed with 10% aqueous citric acid (2×200 mL), and the solvent was removed under vacuum to afford 11.6 g of (R)-2-(1-(tert-butoxycarbonyl)-pyrrolidin-3-yl)acetic acid as an off-white solid (92% yield). ¹H NMR (DMSO-d₆, 400 MHz): δ 12.1 (s, 1H); 3.36-3.48 (m, 1H); 3.20-3.34 (m, 1H); 3.05-3.19 (m, 1H, 2.72-2.84 (m, 1H); 2.30-2.42 (m, 1H), 2.22-2.30 (m, 2H); 1.85-2.00 (m, 1H); 1.38-1.54 (m, 1H), 1.35 (2, 9H).

### Example 8

### Synthesis of tert-butyl (R)-3-(2-hydroxyethyl)pyrrolidine-1-carboxylate (6)

Procedure A:

A solution of (R)-2-(1-(tert-butoxycarbonyl)pyrrolidine-3-yl)acetic acid (49.0 g, 214 mmol) in tetrahydrofuran (THF) (200 mL) was cooled to −10° C. 250 mL (250 mmol) of a 1M borane in THF solution was added slowly to the flask while maintaining the temperature lower than 0° C. The solution was warmed to ambient temperature and stirred for 1 h. The solution was sampled hourly and analyzed by HPLC to establish completion of the reaction. Upon completion of the reaction, the solution was cooled to 0° C., and a 10% sodium hydroxide solution (80 mL) was added drop-wise over a 30 minute period to control gas evolution. The solution was extracted with 500 mL of a 1:1 hexanes/ethyl acetate solution. The organic layer was washed with saturated sodium chloride solution and dried with 10 g of silica gel. The silica gel was removed by filtration and washed with 100 mL of 1:1 hexanes/ethyl acetate. The organic layers were combined and concentrated under vacuum to give 6 (42 g, 91.3%) as a light-orange oil that solidified upon sitting. ¹H NMR (CDCl₃, 400 MHz) δ 3.67 (m, 2H), 3.38-3.62 (m, 2H), 3.25 (m, 1H), 2.90 (m, 1H), 2.25 (m, 1H), 1.98-2.05 (m, 1H), 1.61-1.69 (m, 2H), 1.48-1.59 (m, 2H), 1.46 (s, 9H).

Procedure B:

Borane-THF complex (3.90 kg or L of 1M in THF, mol) was added slowly to a stirred solution of (R)-2-(1-(tert-butoxycarbonyl)pyrrolidine-3-yl)acetic acid (683 g, 3.03 mol) in THF (2.5 kg), kept under nitrogen gas, and using a water bath to keep the temperature between 23 and 28° C. The addition took 1.75 h. Stirring at 25° C. was continued for 1 h, after which time GC analysis indicated complete reaction. The reaction mixture was cooled to <10° C. and maintained below 25° C. as 10% aqueous sodium hydroxide (1.22 kg) was slowly added. The addition took 40 min. The mixture was stirred 1 h at 25° C., and then combined with 1:1 (v/v) heptane/ethyl acetate (7 L). The mixture was stirred for 15 min and allowed to separate into phases (1 h). The organic phase was withdrawn, and the aqueous phase was combined with a second 7 L portion of 1:1 heptane/ethyl acetate. This was stirred for 15 min and allowed to separate into phases (20 min). The organic phase was again withdrawn, and the combined organic phases were washed with saturate aqueous sodium chloride (4.16 kg), using 15 min of mixing and 1 h of settling time. The organic phase was combined with silica gel (140 g) and stirred 1 h. The anhydrous sodium sulfate (700 g) was added, and the mixture was stirred for 1.5 h. The mixture was filtered, and the filter cake was washed with 1:1 heptane/ethyl acetate (2 L). The filtrate was concentrated under vacuum at <40° C. for 6 h. The resulting oil weighed 670 g (103% yield) and contains traces of heptane, but is otherwise identical to previously prepared samples of 6, by NMR analysis.

### Example 9

### tert-butyl (R)-3-(2-(methylsulfonyloxy)ethyl)pyrrolidine-1-carboxylate (7)

Procedure A:

To a solution of tert-butyl (R)-3-(2-hydroxymethyl)pyrrolidine-1-carboxylate (41.0 g, 190 mmol)) was added triethylamine (40 mL) in toluene (380 mL) and cooled to −10° C. Methanesulfonyl chloride (20.0 mL, 256 mmol) was added slowly so as to maintain the temperature around −5 to 0° C. The solution was warmed to ambient temperature and stirred for 1 h. The solution was sampled hourly and analyzed by HPLC to establish completion of the reaction. Upon completion of reaction, the solution was filtered, and the filtrate was washed with a 5% sodium bicarbonate solution (250 mL). The organic layer was collected and washed with a saturated aqueous sodium chloride solution (250 mL). The organic layer was collected, dried over silica gel (10 g), and concentrated under vacuum to give 7 (53.0 g, 92.8%) as a light-yellow viscous oil. ¹H NMR (CDCl₃, 400 MHz) δ 4.26 (t, J=6.8 Hz, 2H), 3.41-3.63 (m, 2H), 3.27 (m, 1H), 3.02 (s, 3H), 2.92 (m, 1H), 2.28 (m, 1H), 2.05 (m, 1H), 1.83 (m, 2H), 1.50-1.63 (m, 1H), 1.46 (s, 9H).

Procedure B:

Under a nitrogen atmosphere, a solution of triethylamine (460 g, 4.55 mol) and tert-butyl (R)-3-(2-hydroxymethyl)pyrrolidine-1-carboxylate (the entire sample from Example 7, Procedure B, 3.03 mol) in toluene (5.20 kg) was stirred and cooled to 5° C. Methanesulfonyl chloride (470 g, 4.10 mol) was added slowly, over a 1.25 h, keeping the temperature below 15° C. using ice bath cooling. The mixture was gradually warmed (over 1.5 h) to 35° C., and this temperature was maintained for 1.25 h, at which point GC analysis indicated that the reaction was complete. The mixture was cooled to 25° C., and solids were filtered off and the filter cake washed with toluene (1.28 kg). The filtrate was stirred with 10% aqueous sodium bicarbonate (4.0 kg) for 15 min, and the phases were allowed to separate for 30 min. The organic phase was then stirred with saturated aqueous sodium chloride (3.9 kg) for 30 min, and the phases were allowed to separate for 20 min. The organic phase was combined with silica gel (160 g) and stirred for 1 h. Anhydrous sodium sulfate (540 g) was added, and the mixture was stirred an additional 40 min. The mixture was then filtered, and the filter cake was washed with toluene (460 g). The filtrate was concentrated under vacuum at 50° C. for 5 h, and the resulting oil was kept under vacuum at 23° C. for an additional 8 h. This left 798 g of 7, 93% pure by GC analysis.

### Example 10

### Synthesis of tert-butyl (R)-3-vinylpyrrolidine-1-carboxylate (9)

Procedure A:

A solution of tert-butyl (R)-3-((methylsulfonyloxy)ethyl)pyrrolidine-1-carboxylate (49.0 g, 167 mmol), sodium iodide (30.0 g, 200 mmol) and 1,2-dimethoxyethane (450 mL) was stirred at 50-60° C. for 4 h. The solution was sampled hourly and analyzed by HPLC to establish completion of the reaction. Upon completion of reaction, the solution was cooled to −10° C., and solid potassium tert-butoxide (32.0 g, 288 mmol) was added while maintaining temperature below 0° C. The reaction mixture was warmed to ambient temperature and stirred for 1 h. The mixture was sampled hourly and analyzed by HPLC to establish completion of the reaction. Upon completion of reaction, the mixture was filtered through a pad of diatomaceous earth (25 g dry basis). The cake was washed with 1,2-dimethoxyethane (100 mL). The combined filtrates were concentrated under vacuum, to yield an orange oil with suspended solids. The oil was dissolved in hexanes (400 mL), stirred for 30 min, and filtered to remove the solids. The organic layer was dried over silica gel (10 g), and concentrated under vacuum to give 9 (26.4 g, 82.9%) as a colorless oil. ¹H NMR (CDCl₃, 400 MHz) δ 5.77 (m, 1H), 5.10 (dd, J=1.2 Hz, J=16 Hz, 1H), 5.03 (dd, J=1.2 Hz, J=8.8 Hz, 1H), 3.41-3.59 (m, 2H), 3.29 (m, 1H), 3.05 (m, 1H), 2.78 (m, 1H), 2.01 (m, 1H), 1.62-1.73 (m, 1H), 1.46 (m, 9H).

Procedure B:

A solution of tert-butyl (R)-3-(2-(methylsulfonyloxy)ethyl)pyrrolidine-1-carboxylate (792 g of the product of Example 7, Procedure B, ˜2.5 mol), sodium iodide (484 g, 3.27 mol) and 1,2-dimethoxyethane (7.2 L) was stirred at 55° C. for 4.5 h under nitrogen, at which time GC analysis indicated that the reaction was complete. The solution was cooled to <10° C., and solid potassium tert-butoxide (484 g, 4.32 mol) was added in portions (1.25 h addition time) while maintaining temperature below 15° C. The reaction mixture was stirred 1 h at 5° C., warmed slowly (6 h) to 20° C., and stirred at 20° C. for 1 h. The solution was filtered through a pad of diatomaceous earth (400 g dry basis). The filter cake was washed with 1,2-dimethoxyethane (1.6 kg). The combined filtrates were concentrated under vacuum, and the semisolid residue was stirred with heptane (6.0 L) for 2 h. The solids were removed by filtration (the filter cake was washed with 440 mL of heptane), and the filtrate was concentrated under vacuum at 20° C. to give 455 g of 9 (90.7% pure). A sample of this material (350 g) was fractionally distilled at 20-23 torr to give 296 g of purified 9 (bp 130-133° C.) (>99% pure by GC analysis).

### Example 11

### Synthesis of (R)-5-((E)-2-pyrrolidin-3-ylvinyl)pyrimidine (11)

Nitrogen was bubbled through a solution of (R)-tert-butyl 3-vinylpyrrolidine-1-carboxylate (25 g, 127 mmol), 5-bromopyrimidine (30.3 g, 190 mmol), 1,1′-bis(diphenylphosphino)ferrocene (2.11 g, 3.8 mmol), and sodium acetate (18.8 gr, 229 mmol) in N,N-dimethylacetamide (250 mL) for 1 h, and palladium acetate (850 mg, 3.8 mmol) was added. The reaction mixture was heated to 150° C. at a rate of 40° C./h and stirred for 16 h. The mixture was cooled to 10° C. and quenched with water (750 mL) while maintaining an internal temperature below 20° C. MTBE (300 mL) was added, followed by diatomaceous earth (40 g, dry basis). The suspension was stirred for 1 h at ambient temperature and filtered through a bed of diatomaceous earth. The residue was washed with MTBE (2×100 mL) and the filtrate was transferred to a 2-L vessel equipped with an overhead stirrer and charged with activated charcoal (40 g). The suspension was stirred for 2 h at ambient temperature and filtered through diatomaceous earth. The residue was washed with MTBE (2×100 mL,), and the filtrate was concentrated in vacuo to afford 28.6 g of an orange oil. The oil is dissolved in MTBE (100 mL) and Si-Thiol® (2.0 g, 1.46 mmol thiol/g, Silicycle Inc.) was added. The suspension was stirred under nitrogen at ambient temperature for 3 h, filtered through a fine filter, and held in a glass container.

To a solution of 6 M HCl (70 mL) was added the filtrate over a period of 30 min while maintaining the internal temperature between 20° C. and 23° C. The mixture was stirred vigorously for 1 h and the organic layer removed. The remaining aqueous layer was basified with 45 wt % KOH (50 mL), and the resulting suspension was extracted once with chloroform (300 mL). Evaporation of the solvent in vacuo (bath temperature at 45° C.) gave 16.0 g (71.8%), of (R)-5-((E)-2-pyrrolidin-3-ylvinyl)pyrimidine free base as a red oil, which is immediately dissolved in isopropanol (50 mL) and used for salt formation.

### Example 12

### Synthesis of (R)-5-((E)-2-pyrrolidin-3-ylvinyl)pyrimidine mono-citrate

To a solution of citric acid (17.6 g, 91.6 mmol) in isopropanol (250 mL) and water (25 mL) was added drop-wise a solution of (R)-5-((E)-2-pyrrolidin-3-ylvinyl)pyrimidine free base (16.0 g, 91.2 mmol) in isopropanol (50 mL) at 55° C. The resulting solution was seeded with (R)-5-((E)-2-pyrrolidin-3-ylvinyl)pyrimidine mono-citrate Form II (200 mg) and stirred for 15 min. The suspension was heated to 65° C. and stirred for 1 h, after which the suspension was cooled to 20° C. at −10° C./h and allowed to stand at 20° C. for 12 h. The suspension was filtered through a coarse glass filter, and the collected solid was washed with isopropanol (64 mL) and methyl tert-butyl ether (64 mL). The resulting, free-flowing, tan solid was dried in vacuo at 70° C. to give 17.4 g (36%) of (R)-5-((E)-2-pyrrolidin-3-ylvinyl)pyrimidine mono-citrate (mixture of Forms II and III) as a tan solid. ¹H NMR (D₂O, 400 MHz) δ: 8.85 (s, 1H), 8.70 (s, 1H), 6.50 (d, J=17 Hz, 1H), 6.35 (dd, J=7 Hz, J=17 Hz, 1H), 3.43-3.50 (m, 1H), 3.34-3.43 (m, 1H), 3.20-3.30 (m, 1H), 3.08-3.19 (m, 1H), 3.00-3.08 (m, 1H), 2.77 (d; J=16 Hz, 2H), 2.65 (d, J=16 Hz, 2H), 2.16-2.26 (m, 1H), 1.80-1.92 (m, 1H).

### Example 13

### Screen for hydrochloric acid addition salts of (R)-5-((E)-2-pyrrolidin-3-ylvinyl)pyrimidine

(R)-5-((E)-2-Pyrrolidin-3-ylvinyl)pyrimidine free base was dissolved in either, isopropyl acetate, tetrahydrofuran, methyl isobutyl ketone, acetonitrile, or isopropyl alcohol. The resulting solution was treated with 1 eq. of HCl delivered in one of the following forms: 1M in diethyl ether, 1M in water, 5M in isopropyl alcohol or 4M in dioxane. The mixture was incubated at 50° C./ambient temperature (4 h cycles) for 24 h. Where the experiment resulted in a stable solid, the material was analyzed by XRPD.

### Example 14

### Screen for “mono” acid addition salts of (R)-5-((E)-2-pyrrolidin-3-ylvinyl)pyrimidine

(R)-5-((E)-2-Pyrrolidin-3-ylvinyl)pyrimidine free base (10 mg, 0.057 mmol) was dissolved in either isopropyl acetate or acetonitrile. The solutions were treated with 1 eq. of the corresponding acid (see below), warmed to 50° C., and cooled slowly to ambient temperature overnight. The solvent was then evaporated under vacuum without heating, and the residues analyzed by XRPD. The solids are then stored in a humidity chamber at 40° C. and 75% RH for a week, and re-analyzed by XRPD.

In the cases where the experiment did not yield a crystalline solid, the samples were maturated in tetrahydrofuran and isopropyl alcohol, and where a solid was obtained, the solid was analyzed by XRPD and stored in the humidity chamber for a week to assess stability.

The following acids were screened, using the above procedures for forming “mono” acid addition salts: hydrochloric acid, sulfuric acid, methanesulfonic acid, maleic acid, phosphoric acid, 1-hydroxy-2-naphthoic acid, ketoglutaric acid, malonic acid, L-tartaric acid, fumaric acid, citric acid, L-malic acid, hippuric acid, L-lactic acid, benzoic acid, succinic acid, adipic acid, acetic acid, nicotinic acid, propionic acid, orotic acid, 4-hydroxybenzoic acid, and di-p-Toluoyl-D-tartaric acid.

### Example 15

### Screen for “hemi” acid addition salts of (R)-5-((E)-2-pyrrolidin-3-ylvinyl)pyrimidine

(R)-5-((E)-2-Pyrrolidin-3-ylvinyl)pyrimidine free base (10 mg, 0.057 mmol) was dissolved in either isopropyl acetate or acetonitrile. The solutions were then treated with 0.5 eq. of the corresponding acid (see below), warmed to 50° C., and cooled slowly to ambient temperature overnight. The solvent was then evaporated under vacuum without heating, and the residues analyzed by XRPD. The solids were then stored in the humidity chamber at 40° C. and 75% RH for a week, and re-analyzed by XRPD.

In the cases where the experiment did not yield a crystalline solid, these samples were maturated in tetrahydrofuran and isopropyl alcohol, and where a solid was obtained, the solid is analyzed by XRPD and stored in the humidity chamber for a week to assess stability.

The following acids were screened, using the above procedures for forming “hemi” acid addition salts: sulfuric acid, maleic acid, phosphoric acid, ketoglutaric acid, malonic acid, L-tartaric acid, fumaric acid, citric acid, L-malic acid, succinic acid, adipic acid, and di-p-toluoyl-D-tartaric acid.

### Example 16

### General scale-up procedure for selected salts of (R)-5-((E)-2-pyrrolidin-3-ylvinyl)pyrimidine

A number of (R)-5-((E)-2-pyrrolidin-3-ylvinyl)pyrimidine salts were chosen to scale-up to ˜200 mg for further characterization. These salt forms include: citrate (mono and hemi), orotate (mono), 4-hydroxybenzoate (mono), di-p-toluoyl-D-tartrate (mono and hemi), maleate (mono and hemi), and fumarate (mono and hemi).

(R)-5-((E)-2-pyrrolidin-3-ylvinyl)pyrimidine free base (189 mg, 1.077 mmol, was dissolved in acetonitrile. The solution was then treated with 1.1 eq. of the corresponding acid for the preparation of the mono salt, and 0.5 eq. for the preparation of the hemi salt. The mixture was warmed up to 50° C. and cooled down slowly to ambient temperature overnight.

The solid obtained was filtered and dried under suction before being analyzed by XRPD, and ¹H-NMR. TGA experiments were performed to determine content of water or other solvents, and DSC experiments were run to establish stability of the isolated forms and the possibility of new forms for each salt. DVS experiments were used to assess hygroscopicity of the salts. HPLC purity and thermodynamic solubility were also measured for each salt.

### Example 17

### (R)-5-((E)-2-Pyrrolidin-3-ylvinyl)pyrimidine mono-citrate Form I

(R)-5-((E)-2-Pyrrolidin-3-ylvinyl)pyrimidine mono-citrate Form I was obtained according to the mono salt screening procedure, from isopropyl acetate, by evaporation and maturation in tetrahydrofuran. Alternatively, the mono-citrate Form I was obtained according to the mono salt screening procedure, from acetonitrile, by evaporation and maturation in isopropyl alcohol.

### Example 18

### (R)-5-((E)-2-Pyrrolidin-3-ylvinyl)pyrimidine mono-citrate Form II

A suspension of the (R)-5-((E)-2-pyrrolidin-3-ylvinyl)pyrimidine mono-citrate Forms II and III mixture in methanol was heated to 50° C. and stirred for 1 h. The suspension was subsequently cooled to 20° C. at a rate of −30° C./h, followed immediately by heating back to 50° C. at a rate of +30° C./h. Heating was discontinued upon reaching 50° C., and the suspension was cooled and stirred at ambient temperature for 16 h. The suspension was filtered, and any residual material in the flask was rinsed out with additional methanol. The residue was dried at 70° C. in vacuo for 16 h to give (R)-5-((E)-2-pyrrolidin-3-ylvinyl)pyrimidine mono-citrate Form II.

### Example 19

### Amorphous (R)-5-((E)-2-pyrrolidin-3-ylvinyl)pyrimidine mono-citrate

Amorphous (R)-5-((E)-2-pyrrolidin-3-ylvinyl)pyrimidine mono-citrate was prepared by freeze drying a solution of (R)-5-((E)-2-pyrrolidin-3-ylvinyl)pyrimidine mono-citrate Form II in water.

### Example 20

### (R)-5-((E)-2-Pyrrolidin-3-ylvinyl)pyrimidine mono-citrate Form III

(R)-5-((E)-2-pyrrolidin-3-ylvinyl)pyrimidine mono-citrate Form III was prepared by allowing amorphous (R)-5-((E)-2-pyrrolidin-3-ylvinyl)pyrimidine mono-citrate to stand at ambient temperature for two hours.

### Example 21

### (R)-5-((E)-2-Pyrrolidin-3-ylvinyl)pyrimidine mono-citrate Form IV

(R)-5-((E)-2-Pyrrolidin-3-ylvinyl)pyrimidine mono-citrate Form IV was obtained by maturation of Form II in acetone/methyl isobutyl ketone.

### Example 22

### (R)-5-((E)-2-Pyrrolidin-3-ylvinyl)pyrimidine mono-(R)-(−)-orotate salt

Orotic acid (0.965 g, 6.18 mmol) was added as a solid to a stirring, hot solution of (R)-5-((E)-2-pyrrolidin-3-ylvinyl)pyrimidine free base (1.084 g, 6.18 mmol) in 2-propanol (10 mL) in a round-bottomed flask. The resulting mixture of solids was heated under reflux for 5 min, cooled to ambient temperature and stirred overnight. The light-beige powder was filtered, washed with 2-propanol (10, 8 mL) and dried in a vacuum oven (air bleed) at 50° C. for 20 h to give 1.872 g (77.9%) of an off-white to white, lumpy solid, mp 230-233° C. ¹H NMR (D₂O): δ 8.80 (s, 1H), 8.60 (s, 2H), 6.40 (d, 1H), 6.25 (dd, 1H), 5.93 (s, 1H, ═CH of orotic acid, indicating a mono-salt stoichiometry), 3.38 (dd, 1H), 3.29 (m, 1H), 3.17 (m, 1H), 3.04 (m, 1H), 2.97 (dd, 1H), 2.13 (m, 1H), 1.78 (m, 1H). Elemental analysis results suggests the presence of excess orotic acid and a 1:1.1 base:orotic acid salt stoichiometry. Elemental Analysis Calculated for C₁₀H₁₃N₃.C₅H₄N₂O₄: (C, 54.38%; H, 5.17%, N, 21.14%). Found: (C, 53.49%, 53.44%; H, 5.04%, 5.10%; N, 20.79%, 20.84%).

### Example 23

### (R)-5-((E)-2-Pyrrolidin-3-ylvinyl)pyrimidine mono-orotate Form I

(R)-5-((E)-2-pyrrolidin-3-ylvinyl)pyrimidine free base (189 mg, 1.077 mmol, freshly prepared) was dissolved in acetonitrile (5 ml). The solution was then treated with 1.1 eq. of an orotic acid solution (1M in ethanol) at ambient temperature. The mixture was warmed up to 50° C. and cooled down slowly to ambient temperature overnight. The solid obtained was filtered and dried under suction before being analyzed by XRPD, and ¹H-NMR.

### Example 24

### (R)-5-((E)-2-Pyrrolidin-3-ylvinyl)pyrimidine mono-maleate Form I

(R)-5-((E)-2-pyrrolidin-3-ylvinyl)pyrimidine free base (189 mg, 1.077 mmol, freshly prepared) was dissolved in acetonitrile (5 ml). The solution was then treated with 1.1 eq. of an maleic acid solution (1M in tetrahydrofuran) at ambient temperature. The mixture was warmed up to 50° C. and cooled down slowly to ambient temperature overnight. The solid obtained was filtered and dried under suction before being analysed by XRPD, and ¹H-NMR.

### Example 25

### (R)-5-((E)-2-Pyrrolidin-3-ylvinyl)pyrimidine mono-maleate Form II

(R)-5-((E)-2-pyrrolidin-3-ylvinyl)pyrimidine mono-maleate (Form I) was slurried in ethanol and incubated at 50° C./r.t. 4 h-cycle for 48 h. XRPD analysis of the solid showed Form II.

### Example 26

### (R)-5-((E)-2-Pyrrolidin-3-ylvinyl)pyrimidine mono-oxalate

Oxalic acid (0.516 g, 5.73 mmol) was added as a solid to a stirring, warm solution of (R)-5-((E)-2-pyrrolidin-3-ylvinyl)pyrimidine (1.00 g, 5.70 mmol) in ethanol (10 mL). The salt precipitated upon further warming of the solution. To facilitate stirring, the mixture was diluted with ethanol (6 mL), and the lumps were broken with a spatula. The mixture was cooled to ambient temperature and was left standing overnight. The light-beige powder was filtered, washed with ethanol, and dried in a vacuum oven at 50° C. for 6 h to give 1.40 g (92.3%) of a creamy-white, fluffy powder, mp 149-151° C. ¹H NMR (DMSO-d₆): δ 9.03 (s, 1H), 8.86 (s, 2H), 6.56 (m, 2H), 3.40 (dd, 1H), 3.31 (m, 1H), 3.18 (m, 1H), 3.08 (m, 1H), 2.96 (dd, 1H), 2.15 (m, 1H), 1.80 (m, 1H), ¹³C NMR (DMSO-d₆): δ 164.90 (C═O of oxalic acid), 156.97, 154.17, 133.66, 130.31, 124.20, 48.70, 44.33, 40.98, 30.42. Elemental analysis: Calculated for C₁₀H₁₃N₃.C₂H₂O₄ (C, 54.33%; H, 5.70%, N, 15.84%). Found (C, 54.39%, 54.29%; H, 5.68%, 5.66%; N, 15.68%, 15.66%).

### Example 27

### (R)-5-((E)-2-Pyrrolidin-3-ylvinyl)pyrimidine hemi-di-p-toluoyl-D-tartarate

Solid di-p-toluoyl-D-tartarate salts was obtained according to the “hemi” salt screening procedure from isopropyl acetate or acetonitrile by evaporation, or by evaporation if isopropyl acetate followed by maturation with tetrahydrofuran or by evaporation of acetonitrile followed by maturation with isopropyl alcohol.

The following procedure was used to make a larger quantity of the salt. (+)-O,O′-Di-p-toluoyl-D-tartaric acid (1.103 g, 2.85 mmol) was added as a solid to a stirring, warm solution of (R)-5-((E)-2-pyrrolidin-3-ylvinyl)pyrimidine free base (1.007 g, 5.74 mmol) in ethanol (10 mL). A few insoluble solids precipitated that failed to dissolve upon heating the mixture to reflux. The light amber solution (with a few fine solids) was stirred for 4-5 h and then allowed to stand at ambient temperature overnight. The precipitation of the salt as a light beige powder was slow. After stirring for 15 days, the solids were filtered, washed with ethanol (5 mL) and dried in a vacuum oven at 50° C. for 21 h to give 1.50 g (71.5%) of an off-white to slightly yellow-tinged powder, mp 178-180° C. ¹H NMR (DMSO-d₆) confirms the 1:0.5 base:acid salt stoichiometry. ¹H NMR (DMSO-d₆): δ 10.30 (broad s, ˜1H), 9.02 (s, 1H), 8.80 (s, 2H), 7.87 (d, 2H, —C₆H₄—, indicating a hemi-salt stoichiometry), 7.27 (d, 2H, —C₆H₄—, indicating a hemi-salt stoichiometry), 6.40 (dd, 1H), 6.34 (d, 1H), 5.58 (s, 1H, CH(CO₂H)—O— of acid moiety, indicating a hemi-salt stoichiometry), 3.21 (dd, 1H), 3.14 (m, 1H), 3.00 (m, 1H), 2.86 (m, 1H), 2.75 (dd, 1H), 2.30 (s, 3H, —CH₃ of acid moiety, indicating a hemi-salt stoichiometry), 1.93 (m, 1H), 1.61 (m, 1H).

### Example 28

### (R)-5-((E)-2-Pyrrolidin-3-ylvinyl)pyrimidine hemi-di-p-benzoyl-D-tartarate

(+)-O,O′-Di-benzoyl-D-tartaric acid (1.025 g, 2.72 mmol) was added as a solid to a stirring, warm solution of (R)-5-((E)-2-pyrrolidin-3-ylvinyl)pyrimidine free base (1.003 g, 5.72 mmol) in ethanol (10 mL). The mixture was heated to near reflux on a hot plate, producing a light amber solution. The resulting solution was cooled to ambient temperature and was left standing overnight. Because no solids were present, the solution was slowly evaporated in a fume hood, affording tan-brown, gummy solids. Isopropyl acetate (10 mL) was added and with spatula scraping and stirring, light beige solids are deposited. The mixture was stirred overnight. The solids were filtered, washed with isopropyl acetate (2×5 mL) and dried in a vacuum oven at 50° C. for 24 h to give 1.93 g (95.2%) of an off-white powder, mp 155-160° C. ¹H NMR (DMSO-d₆) confirmed the 1:0.5 base:acid salt stoichiometry. ¹H NMR (DMSO-d₆): δ 10.25 (broad s, ˜1H), 9.02 (s, 1H), 9.80 (s, 2H), 7.98 (d, 2H C₆H₅—), 7.57 (m, 1H, C₆H₅—), 7.48 (m, 2H, C₆H₅—), 6.38 (m, 2H), 5.61 (s, 1H, —CH(CO₂H)—O— of acid moiety, indicating a hemi-salt stoichiometry), 3.22 (dd, 1H), 3.14 (dt, 1H), 3.00 (dt, 1H), 2.88 (m, 1H), 2.77 (dd, 1H), 1.92 (m, 1H), 1.61 (m, 1H).

### Example 29

### (R)-5-((E)-2-Pyrrolidin-3-ylvinyl)pyrimidine hemi-di-p-anisoyl-D-tartarate

(+)-Di-p-anisoyl-D-tartaric acid (1.199 g) was added as a solid to a stirring, warm solution of (R)-5-((E)-2-pyrrolidin-3-ylvinyl)pyrimidine free base (0.999 g) in ethanol (10 mL). The resulting solution, with a few solids present, was stirred and heated in an attempt to dissolve all solids. The solution became a thick mass. After standing at ambient temperature for 4-5 h, additional ethanol (10 mL) was added. The mixture containing light-beige to cream-colored solids was stirred overnight. The solids were filtered, washed with ethanol (10 mL), and dried in a vacuum oven at 50° C. for 21 h to give 1.91 g (87.3%) of a white powder, mp 173-177° C. ¹H NMR (DMSO-d₆) confirmed the 1:0.5 base:acid salt stoichiometry. ¹H NMR (DMSO-d₆): δ 10.20 (broad s, ˜1H), 9.02 (s, 1H), 8.80 (s, 2H), 7.93 (d, 2H, —C₆H₄—, indicating a hemi-salt stoichiometry), 7.00 (d, 2H, —C₆H₄—, indicating a hemi-salt stoichiometry), 6.40 (dd, 1H), 6.34 (d, 1H), 5.56 (s, 1H, CH(CO₂H)—O— of acid moiety, indicating a hemi-salt stoichiometry), 3.76 (s, 3H, —OCH₃ of acid moiety, indicating a hemi-salt stoichiometry), 3.22 (dd, 1H), 3.14 (m, 1H), 3.01 (m, 1H), 2.85 (m, 1H), 2.75 (m, 1H), 1.92 (m, 1H), 1.61 (m, 1H).

### Example 30

### (R)-5-((E)-2-Pyrrolidin-3-ylvinyl)pyrimidine mono-di-p-toluoyl-D-tartarate

Solid di-p-toluoyl-D-tartarate salts were obtained according to the “mono” salt screening procedure from isopropyl acetate or acetonitrile by evaporation.

The following procedure was used to make a larger quantity of the salt. (+)-O,O′-Di-p-toluoyl-D-tartaric acid (2.205 g, 5.71 mmol) was added as a solid to a stirring, warm solution of (R)-5-((E)-2-pyrrolidin-3-ylvinyl)pyrimidine free base (1.000 g, 5.70 mmol) in ethanol (21 mL). Precipitation of the salt was immediate. After gently heating the stirring mixture on a hot plate to near reflux, the resulting mixture was cooled to ambient temperature. The resulting solids were filtered, washed with ethanol (3×5 mL), and dried in a vacuum oven at 50° C. for 13 h to give 3.081 g (96.1%) of a light-beige powder, mp 181-184° C. ¹H NMR (DMSO-d₆) confirmed the 1:1 salt stoichiometry. ¹H NMR (DMSO-d₆): δ 9.60 (broad s, ˜1H), 9.03 (s, 1H), 8.82 (s, 2H), 7.83 (d, 4H, —C₆H₄—, indicating a mono-salt stoichiometry), 7.27 (d, 4H, —C₆H₄—, indicating a mono-salt stoichiometry), 6.44 (d, 2H), 5.62 (s, 2H, CH(CO₂H)—O— of acid moiety, indicating a mono-salt stoichiometry), 3.30 (dd, 1H), 3.23 (m, 1H), 3.09 (m, 1H), 2.95 (m, 1H), 2.85 (dd, 1H), 2.33 (6H, —CH₃ of acid moiety, indicating a mono-salt stoichiometry), 2.02 (m, 1H), 1.69 (m, 1H).

### Example 31

### (R)-5-((E)-2-Pyrrolidin-3-ylvinyl)pyrimidine mono-di-p-benzoyl-D-tartarate

(+)-O,O′-Di-benzoyl-D-tartaric acid (2.05 g, 5.72 mmol) was added as a solid to a stirring, warm solution of (R)-5-((E)-2-pyrrolidin-3-ylvinyl)pyrimidine free base (0.999 g, 5.69 mmol) in ethanol (21 mL) in a round-bottomed flask, producing a solution. After stirring and further heating, precipitation of the salt occurred in the warm solution. The resulting mixture was cooled to ambient temperature over a two-day weekend. The resulting solids were filtered on a Büchner funnel, washed with ethanol (4×5 mL), and dried in a vacuum oven (air bleed) at 50° C. for 13 h to give 2.832 g (93.0%) of a light-beige to off-white powder, mp 165-171° C. ¹H NMR (DMSO-d₆) confirmed the 1:1 salt stoichiometry. ¹H NMR (DMSO-d₆): δ 9.65 (broad s, ˜1H), 9.03 (s, 1H), 9.83 (s, 2H), 7.94 (d, 4H, C₆H₅—), 7.60 (m, 2H, C₆H₅—), 7.50 (m, 4H, C₆H₅—), 6.45 (m, 2H), 5.67 (s, 2H, —CH(CO₂H)—O— of acid moiety, indicating a mono-salt stoichiometry), 3.31 (dd, 1H), 3.22 (m, 1H), 3.08 (m, 1H), 2.96 (m, 1H), 2.85 (dd, 1H), 2.01 (m, 1H), 1.69 (m, 1H).

### Example 32

### (R)-5-((E)-2-Pyrrolidin-3-ylvinyl)pyrimidine mono-(1S)-10-camphorsulfonate

(1S)-(+)-10-Camphorsulfonic acid (1.329 g, 5.72 mmol) was added as a solid to a stirring, warm solution of (R)-5-((E)-2-pyrrolidin-3-ylvinyl)pyrimidine free base (1.00 g) in 2-propanol (23 mL, 5.70 mmol) in a round-bottomed flask. Upon cooling to ambient temperature, there was no precipitation of solids. The solution was allowed to stand overnight. Gelatinous material containing white solids was observed. After stirring two days, the mixture was diluted with 2-propanol (10.5 mL) because stirring this jelly-like white mass was difficult. After overnight stirring, the resulting white powder was filtered on a Büchner funnel, washed with 2-propanol (5 mL) (NOTE: The solids appeared to have some solubility in 2-propanol) and dried in a vacuum oven (air bleed) at 50° C. for 6 h to give 1.47 g (63.2%) of a white powder, mp 172-173° C. ¹H NMR (DMSO-d₆) confirms the 1:1 salt stoichiometry. After standing seven days, a second crop of light-beige needles was observed in the crystallization liquors. This material was filtered, washed with 2-propanol (10 mL) and dried in a vacuum oven (air bleed) at 50° C. for 21 h to give 0.245 g of light-beige needles, mp 173-174° C. ¹H NMR (DMSO-d₆): δ 9.03 (s, 1H), 8.87 (s, 2H), 6.57 (m, 2H), 3.41 (dd, 1H) 3.33 (m, 1H, partially masked by H₂O), 3.21 (m, 1H), 3.10 (m, 1H), 2.98 (dd, 1H), 2.89 (d, 1H, —CH₂— of acid moiety, indicating a mono-salt stoichiometry), 2.64 (m, 1H), 2.41 (d, 1H, —CH₂— of acid moiety, indicating a mono-salt stoichiometry), 2.25 (t, 0.5H), 2.20 (t 0.5H), 2.15 (m, 1H), 1.93 (t, 1H), 1.82 (m, 3H), 1.28 (m, 2H, —CH₂— of acid moiety, indicating a mono-salt stoichiometry), 1.03 (s, 3H, —CH₃ of acid moiety, indicating a mono-salt stoichiometry), 0.73 (s, 3H, —CH₃ of acid moiety, indicating a mono-salt stoichiometry).

### Example 33

### (R)-5-((E)-2-Pyrrolidin-3-ylvinyl)pyrimidine mono-(1R,2S)-(+)-Camphorate

(1R,2S)-(+)-Camphoric acid (1.149 g, 5.74 mmol) was added as a solid to a stirring, warm solution of (R)-5-((E)-2-pyrrolidin-3-ylvinyl)pyrimidine free base (1.00 g, 5.70 mmol) in ethanol (14 mL) in a round-bottomed flask. Upon heating, all solids dissolved, affording a yellow solution. No precipitate forms upon standing at ambient temperature overnight. The solution was concentrated via rotary evaporation to an amber-brown foam that was dried under vacuum at 50° C. (air bleed) for 6 h to give 2.098 g of a viscous, amber oil. Isopropyl acetate (10 mL) was added, and the solution was allowed to stand at ambient temperature overnight. There was some evidence of crystal nucleation in the gummy, red-amber oil. More isopropyl acetate (10 mL) and 2-propanol (20 drops) was added, and the mixture was gently heated and stirred over 48 h. The resulting milky, creamy solids with some orange lumps were broken with a spatula, and the mixture (colorless liquor) was stirred overnight. The off-white solids were filtered on a Büchner funnel, washed with cold isopropyl acetate (10 mL) and dried in a vacuum oven (air bleed) at 50° C. for 21 h to give 2.034 g (94.9%) of an off-white to cream colored powder, mp 157-159° C. ¹H NMR (DMSO-d₆) confirmed the 1:1 salt stoichiometry. ¹H NMR (DMSO-d₆): δ 9.00 (s, 1H), 8.85 (s, 2H), 6.58 (dd, 1H), 6.47 (d, 1H), 3.17 (dd, 1H), 3.08 (m, 1H), 2.97 (m, 1H), 2.92 (dd, 1H) 2.74 (dd, 1H), 2.61 (dd, 1H), 2.30 (sextet, 1H), 2.00 (m, 2H), 1.65 (m, 2H), 1.32 (m, 1H), 1.15 (s, 3H, —CH₃ of acid moiety, indicating a mono-salt stoichiometry), 1.07 (s, 3H, —CH₃ of acid moiety, indicating a mono-salt stoichiometry), 0.75 (s, 3H, —CH₃ of acid moiety, indicating a mono-salt stoichiometry).

### Example 34

### (R)-5-((E)-2-Pyrrolidin-3-ylvinyl)pyrimidine mono-di-p-anisoyl-D-tartarate

(+)-Di-p-anisoyl-D-tartaric acid (2.388 g, 5.71 mmol) was added as a solid to a stirring, warm solution of (R)-5-((E)-2-pyrrolidin-3-ylvinyl)pyrimidine free base (1.008 g, 5.75 mmol) in ethanol (22 mL) in a round-bottomed flask. Precipitation of the salt occurred before all of the (+)-di-p-anisoyl-D-tartaric acid had been added. The salt did not dissolve upon heating, but the appearance of the solids changed, with conversion to a light, fluffy, white powder. The mixture was cooled to ambient temperature and was stirred over 48 h. The resulting solids were filtered on a Büchner funnel, washed with ethanol (5×5 mL) and dried in a vacuum oven (air bleed) at 50° C. for 13 h to give 3.20 g (94.4%) of an off-white to white, chalky powder, mp 173-176° C. ¹H NMR (DMSO-d₆) confirms the 1:1 salt stoichiometry. ¹H NMR (DMSO-d₆): δ 9.65 (broad s, ˜1H), 9.03 (s, 1H), 8.82 (s, 2H), 7.89 (d, 4H, —C₆H₄—, indicating a mono-salt stoichiometry), 7.01 (d, 4H, —C₆H₄—, indicating a mono-salt stoichiometry), 6.44 (m, 2H), 5.60 (s, 2H, CH(CO₂H)—O— of acid moiety, indicating a mono-salt stoichiometry), 3.79 (s, 6H, —OCH₃ of acid moiety, indicating a mono-salt stoichiometry), 3.30 (dd, 1H), 3.22 (m, 1H), 3.09 (m, 1H), 2.95 (m, 1H), 2.84 (m, 1H), 2.01 (m, 1H), 1.69 (m, 1H).

### Example 35

### (R)-5-((E)-2-Pyrrolidin-3-ylvinyl)pyrimidine mono-(R)-(−)-Phencyphos salt

(R)-(−)-Phencyphos (1.391 g, 5.77 mmol) was added as a solid to a stirring solution of (R)-5-((E)-2-pyrrolidin-3-ylvinyl)pyrimidine free base (1.006 g, 5.73 mmol) in ethanol (10 mL) in a round-bottomed flask. Most of the solids dissolved upon stirring at ambient temperature, and all solids dissolved with gentle heating. The stirring, amber solution was heated to reflux, cooled to ambient temperature and was allowed to stand overnight. The resulting white, needle-like crystals were filtered on a Büchner funnel, washed with cold ethanol (5 mL) and dried in a vacuum oven (air bleed) at 50° C. for 18 h to give 0.811 g (33.9%) of off-white crystals, mp 197-201° C. ¹H NMR (DMSO-d₆) confirms the 1:1 salt stoichiometry. ¹H NMR (DMSO-d₆): δ 9.81 (broad s, ˜1H), 9.02 (s, 1H), 8.85 (s, 2H), 7.27 (m, 5H, C₆H₅—), 6.56 (dd, 1H), 6.48 (d, 1H), 5.00 (d, 1H, —O—CH— of acid moiety, indicating a mono-salt stoichiometry), 4.00 (d, 1H, —O—CH₂— of acid moiety, indicating a mono-salt stoichiometry), 3.48 (dd, 1H, —O—CH₂— of acid moiety, indicating a mono-salt stoichiometry), 3.36 (dd, 1H), 3.30 (m, 1H), 3.17 (m, 1H), 3.07 (m, 1H), 2.93 (dd, 1H), 2.12 (m, 1H), 1.78 (m, 1H), 0.79 (s, 3H, —CH₃ of acid moiety, indicating a mono-salt stoichiometry), 0.60 (s, 3H, —CH₃ of acid moiety, indicating a mono-salt stoichiometry).

### Example 36

### Enteric Formulation of (R)-5-((E)-2-pyrrolidin-3-ylvinyl)pyrimidine in IBS

A trial was conducted using 5 mg Compound A capsules. The capsules made from 2 sub batches (total 120 units) were pooled and characterized to generate batch release data. The batch was characterized in terms of cover appearance, ID, assay (90-110%), related substances (as per the uncoated capsule CofA), and dissolution (USP specs for delayed release).

A short-term stability study was conducted on the coated active batch to support a shelf-life to cover the intended dosing period. Capsules were stored in bulk in 6 glass bottles or HDPE containers (10 units per container) under controlled ambient conditions (15-25° C.) with analysis at 2 time points (e.g. 7 and 35 days). These data were used to support a shelf-life to cover the total time between coating and dosing.

In addition, a short-term ‘in use’ stability study was conducted to support a shelf-life to cover the patient packaging for the product, e.g. Pharmadose or Venalink Cold Seal configurations, for the intended dosing period. Capsules were stored under controlled ambient conditions (15-25° C.) with analysis at 1 time point (e.g. 8 days).

Pharmacopeia disintegration method (as per USP <701>) was used for initial characterization of the acid resistance of the enteric coated capsule formulation. Also, the Pharmacopeia two stage acid-buffer dissolution test for enteric coated Compound A capsules (as per USP <711>) was performed, where capsules are subjected to acidic conditions to demonstrate that the coat holds in place, and then are transferred to a pH 6.8 media to show release of the dose from the enteric coat. The dissolution media was selected as appropriate to ensure that sink conditions were achieved.

### Example 37

### Binding Results for (R)-5-((E)-2-pyrrolidin-3-ylvinyl)pyrimidine

Compound A is an agonist of α₄β₂ and α₃β₄ nueronal nicotinic receptors. In human α₄β₂, Compound A demonstrates a Ki of 17 nM, and in rat α₄β₂ a Ki of 34 nM.

### Cell Lines

Sh-ep1/human α4β2 (Eaton et al., 2003), sh-ep1/human α4β4 (Gentry et al., 2003), sh-ep1/α6β3β4α5 (Grinevich et al., 2005), te671/rd and sh-sy5y cell lines (obtained from Dr. Ron Lukas, Barrow Neurological Institute, St. Joseph's Hospital and Medical Center, Phoenix, Ariz.) were maintained in proliferative growth phase in Dulbecco's modified Eagle's medium (Gibco/brl) with 10% horse serum (Gibco brl), 5% fetal bovine serum (Hyclone, Logan Utah), 1 mm sodium pyruvate, 4 mm I-glutamine. For maintenance of stable transfectants, the α4β2 and α4β4 cell media was supplemented with 0.25 mg/ml zeocin and 0.13 mg/ml hygromycin B. Selection was maintained for the α6β3β4α5 cells with 0.25 mg/ml of zeocin, 0.13 mg/ml of hygromycin B, 0.4 mg/ml of geneticin, and 0.2 mg/ml of blasticidin.

HEK/human α7/RIC3 cells (obtained from J. Lindstrom, U. Pennsylvania, Philadelphia, Pa.) were maintained in proliferative growth phase in Dulbecco's modified Eagle's medium (Gibco/brl) with 10% fetal bovine serum (Hyclone, Logan Utah), 1 mm sodium pyruvate, 4 mm I-glutamine, 0.4 mg/ml geneticin; 0.2 mg/ml hygromycin B.

### Receptor Binding Assays

Preparation of Membranes from Rat Tissues.

Rat cortices were obtained from analytical biological services, incorporated (ABS, Wilmington, Del.). Tissues were dissected from female Sprague-Dawley rats, frozen and shipped on dry ice. Tissues were stored at −20° C. until needed for membrane preparation. Cortices from 10 rats were pooled and homogenized by polytron (Kinematica gmbh, Switzerland) in 10 volumes (weight:volume) of ice-cold preparative buffer (KCl, 11 mM; KH₂PO₄, 6 mM; NaCl 137 mM; Na₂HPO₄ 8 mM; HEPES (free acid), 20 mM; iodoacetamide, 5 mM; EDTA, 1.5 mM; 0.1 mM PMSF pH 7.4). The resulting homogenate was centrifuged at 40,000 g for 20 minutes at 4° C. and the resulting pellet was re-suspended in 20 volumes of ice-cold water. After 60-minute incubation at 4° C., a new pellet was collected by centrifugation at 40,000 g for 20 minutes at 4° C. The final pellet was re-suspended in preparative buffer and stored at −20° C. On the day of the assay, tissue was thawed, centrifuged at 40,000 g for 20 minutes and then resuspended in PBS (Dulbecco's phosphate buffered saline, Life Technologies, pH 7.4) to a final concentration of 2-3 mg protein/ml. Protein concentrations were determined using the Pierce BCA protein assay kit (Pierce Biotechnology, Rockford, Ill.), with bovine serum albumin as the standard.

Preparation of Membranes from Clonal Cell Lines.

Cells were harvested in ice-cold pbs, ph 7.4, then homogenized with a Polytron (Brinkmann Instruments, Westbury, N.Y.). Homongenates were centrifuged at 40,000 g for 20 minutes (4° C.). The pellet was resuspended in PBS and protein concentration determined using the Pierce BCA protein assay kit (Pierce Biotechnology, Rockford, Ill.).

Competition Binding to Receptors in Membrane Preparations.

Binding to nicotinic receptors was assayed on membranes using standard methods adapted from published procedures (Lippiello and Fernandes, 1986; Davies et al., 1999). In brief, membranes were reconstituted from frozen stocks (approximately 0.2 mg protein) and incubated for 2 h on ice in 150 ml assay buffer (PBS) in the presence of competitor compound (0.001 nM to 100 mM) and radioligand. [³H]-nicotine (L-(−)-[N-methyl-3H]-nicotine, 69.5 Ci/mmol, Perkin-Elmer Life Sciences) was used for human α4β2 binding studies. [³H]-epibatidine (52 Ci/mmol, Perkin-Elmer Life Sciences) was used for binding studies at the other receptor subtypes. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times and the radioactivity retained was determined by liquid scintillation counting.

Binding Data Analysis.

Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Kᵢ was calculated using the Cheng-Prusoff equation (Cheng and Prusoff, 1973).

### Example 38

### (R)-5-((E)-2-pyrrolidin-3-ylvinyl)pyrimidine in IBS

Nicotinic α₄β₂-mediated pharmacological effects have been described in neurons that project from the dorsal motor vagal nucleus to various sections of the gut. In the autonomic nervous system, α₃β₄ receptors appear to be present on enterochromaffin cells. Agonists targeting these subtypes would likely ameliorate pathological states where GI motility is compromised. Reference is made to Lee K, Miwa S, Koshimura K, Ito A. Characterization of nicotinic acetylcholine receptors on cultured bovine adrenal chromaffin cells using modified L-[3H]nicotine binding assay. Naunyn Schmiedebergs Arch Pharmacol. 1992; 345(4):363-9; and Racke K, Schworer. Nicotinic and muscarinic modulation of 5-hydroxytryptamine (5-HT) release from procine and canine small intestine. Clin Investig. 1992; 70:190-200.

Preclinically, in two animal models of neuropathic pain, repeat administration of Compound A produced significant analgesia. More specifically, Compound A demonstrates effective analgesia in the Streptozotocin-induced diabetic allodynia model and the Chemotherapy induced model of neuropathic pain. With reference to WO 08/157,365, the rat Streptozotocin-induced diabetic neuropathy model is a clinically relevant model of diabetic neuropathy, which replicates elements of the human situation diabetic condition such as high glucose levels, neuropathic pain in the extremities, and generally poor health. This study demonstrated progressive pain sensitivity, as measured by allodynia testing of the hindpaw at Weeks 4 and 6, and significant reversal of this pain at Week 6 by the test article, in the absence of any changes to blood glucose levels in these groups. The insulin-treated group did show reduced blood glucose levels but did not have significant improvement in pain sensitivity compared in comparison with vehicle-treated animals. This demonstrates a lack of correspondence between blood glucose levels and allodynia levels in this diabetic neuropathy model, and is consistent with reports in the literature (Maneuf, et al, 2004, herein incorporated by reference with regard to such model). The results demonstrated in this study are indicative of Compound A at all three doses tested in the STZ-rat model of diabetic neuropathy. Further, a study of Taxol®-induced neuropathy demonstrated an analgesic effect of the chronically administered test compound (Compound A) as well as acutely administered Gabapentin. Notably, at the 4 week allodynia assessment, the vehicle allodynia response had dropped compared to the 3 week assessment indicating a greater degree of allodynia from which alleviation could be demonstrated. Thus, at three weeks following Taxol®, significant reversal of allodynia demonstrated by the vehicle group was achieved at a 50% threshold of about 10 g force whereas only about 7.5 g was required by week 4.

With analgesia noted from animal models, in a single (SRD) and multiple rising dose (MRD) studies conducted in normal healthy subjects, nausea, vomiting and diarrhea were the most commonly observed adverse events. In the MRD study, following doses 10 mg administered 4 times daily, nausea and vomiting typically occurred within 30 min of dosing—prior to significant systemic absorption. Diarrhea was typically observed more than 5 hours following dosing, when systemic concentrations were approximately 20% of Cₘₐₓ values. It was observed that Compound A has a systemic (plasma) half-life of ˜1 hour or less. Such pharmacodynamic GI motility responses appear to correlate primarily with local effects (possibly agonism of α3β4 receptors located on enterochromaffin cells) rather than systemic plasma concentrations. Thus, an increase in GI motility was also observed in the healthy human subjects such that the pharmacology of Compound A may help alleviate the constipation, pain, and bloating associated with constipation-predominant IBS.

Delivering Compound A selectively to the lower GI tract via enteric coating (EC) may relieve symptoms of IBS-C while avoiding emesis and nausea that can result from drug exposure in the upper GI tract.

### Study Design

A randomized, double-blind, placebo controlled, parallel group, proof of principle study to evaluate the safety, tolerability, and efficacy of Compound A in the treatment of Constipation-Predominant Irritable Bowel Syndrome was conducted.

The objectives of the study include the following: to assess the efficacy of Compound A in the treatment of Constipation Predominant Irritable Bowel Syndrome (IBS-C); and to assess the safety, tolerability and pharmacokinetic profile of Compound A in subjects with IBS-C when administered as a enteric coated capsule for a period of 28 days.

Subjects with constipation predominant irritable bowel syndrome (IBS-C) defined by: the ROME III criteria as: recurrent abdominal pain or discomfort at least 3 days per month, during the previous 3 months that is associated with 2 or more of the following: (1) relieved by defecation; (2) onset associated with a change in stool frequency; and/or (3) onset associated with a change in stool appearance.

Treatment included 5 mg daily (as Enteric Coated capsule) for 14 days followed by 5 mg BID for another 14 days. One endpoint includes global IBS symptom relief (7-point scale). Another endpoint includes bowel movement frequency, pain, bloating, straining, stool consistency, and frequency of rescue medication (laxative). Statistically, a was set to 0.1.

### Results

Compound A was generally well tolerated. All adverse events were mild to moderate. The most commonly reported adverse events were headache or gastrointestinal.

Compound A produces robust increases in spontaneous bowel movements (SBM) that are maintained over at least 1 month but appears to lack analgesic properties. The sample size in this study was not large enough to detect subjective changes. At week 1, straining (p=0.024), pain (p=0.059) and Global Relief Score (p=0.101) all favored Compound A.

Compound A may be primarily a colonic motility enhancer, the results of the study suggests it could be the preferred agent in chronic idiopathic constipation (CIC) with a different mechanism of action, as pain not a primary component in CIC. Compound A may be combined with pain relief as augmentation therapy for IBS-C.

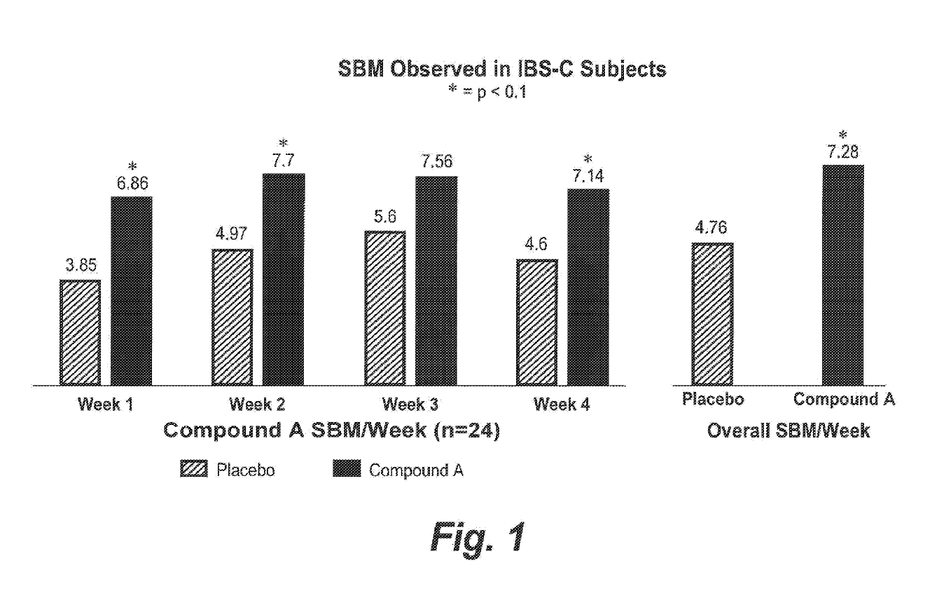

FIG. 1 is a bar graph representation of SBM observed in IBS-C subjects. The left portion of the figure demonstrates objective count of SBM on a weekly basis. The right portion of the figure illustrates the efficacy of Compound A compared to placebo across the entire 4 week treatment period.

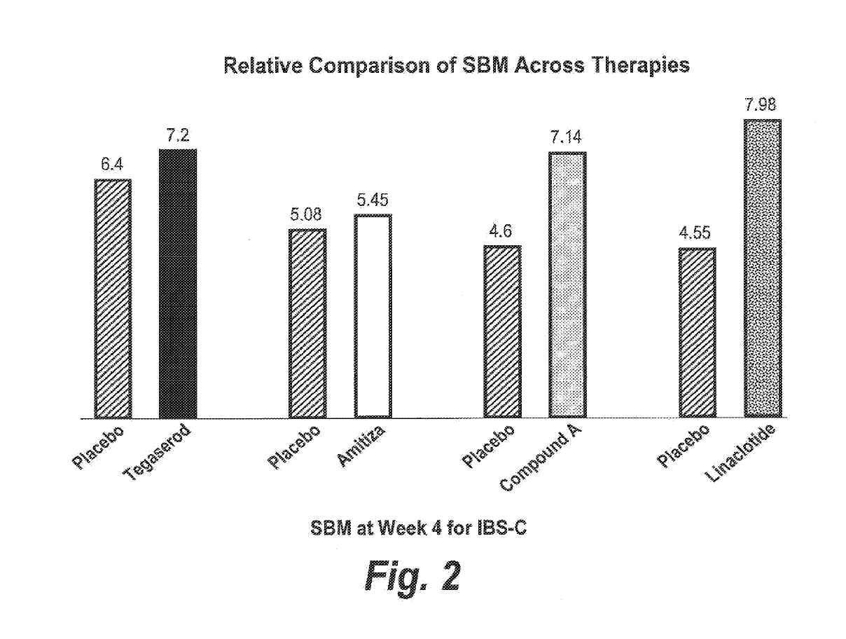

FIG. 2 is a bar graph representation of a relative comparison of SBM across several therapeutics. Compound A was compared with existing and proposed therapies, namely Tegaserod (previously sold under the brand name Zelnorm®, currently withdrawn), Lubiprostone (sold under the trade name Amitiza®), and Linaclotide (currently in Phase III clinical trials), as well as placebo in each case. As illustrated, Compound A compares favorably in SBM at week 4 in subjects with IBS-C.

Data for Tegaserod (previously sold under the brand name Zelnorm®, currently withdrawn) was obtained from NDA Application No. 021200, with particular attention to the Summary of the Basis for Approval. Data for Lubiprostone (sold under the trade name Amitiza®) was obtained from NDA Application No. 021908, with particular attention to the Summary of the Basis for Approval. Data for Linaclotide (currently in Phase III clinical trials) was obtained from Johnston et al., Linaclotide Improves Abdominal Pain and Bowel Habits in a Phase IIb Study of Patients with Irritable Bowel Syndrome with Constipation, Gastronenterology 2010; 139:1877-1886, Lembo et al., Efficacy of Linaclotide for Patients with Chronic Constipation, Gastroenterology 2010; 138(3):886-895 e1, and Lembo et al., Efficacy and Safety of Once Daily Linaclotide Administered Orally for 12 Weeks in Patients with Chronic Constipation: Results from Two Randomized, Double-Blind, Palcebo-Controlled Phase 3 Trials, Presented at Digestive Disease Week (DDW) 2010 in New Orleans, La. 2010; Abstract 286. Each of these references is incorporated by reference with regard to the data provided in FIG. 2, as well as the source of that data.

As herein noted, a total dose of 5 mg (or <100 μg/kg) demonstrates efficacy. One likely efficacious dose for this will be 10 μg/kg<dose<100 μg/kg.

Test compounds were employed in free or salt form. If not otherwise noted, the test substance, Compound A, is provided as its hemigalactarate salt, a white powder.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method for relieving constipation comprising administration of (R)-5-((E)-2-pyrrolidin-3-ylvinyl)pyrimidine or a pharmaceutically acceptable salt thereof.

2. The method of claim 1, wherein the constipation is gastrointestinal, endrocrinological, neurological, iatrogenic, associated with an eating disorder, or related to an injury.

3. A method for treating constipation associated with a gastrointestinal disorder comprising administration of (R)-5-((E)-2-pyrrolidin-3-ylvinyl)pyrimidine or a pharmaceutically acceptable salt thereof.

4. The method of claim 3, wherein the gastrointestinal disorder is irritable bowel syndrome, constipation predominant irritable bowel syndrome, alternating irritable bowel syndrome, chronic idiopathic constipation, acute constipation, drug-induced constipation, colonic disorders, colon cancer, or ileus paralyticus.

5. The method of claim 4, wherein the drug-induced constipation is opioid-induced constipation, antidepressant-induced constipation, antacid-induced constipation, or iron supplement-induced constipation.

6. A method for treating constipation from an endocrinological source comprising administration of (R)-5-((E)-2-pyrrolidin-3-ylvinyl)pyrimidine or a pharmaceutically acceptable salt thereof.

7. The method of claim 6, wherein the endocrinological source is pregnancy or hypothyroidism.

8. A method for treating constipation associated with a neurological condition comprising administration of (R)-5-((E)-2-pyrrolidin-3-ylvinyl)pyrimidine or a pharmaceutically acceptable salt thereof.

9. The method of claim 8, wherein the neurological condition is Parkinson's Disease, multiple sclerosis, or depression.

10. A method for treating constipation associated with an eating disorder comprising administration of (R)-5-((E)-2-pyrrolidin-3-ylvinyl)pyrimidine or a pharmaceutically acceptable salt thereof.

11. A method for treating constipation associated with surgery, spinal cord injury, autonomic dysfunction, paraplegia, age, or long-term patient care comprising administration of (R)-5-((E)-2-pyrrolidin-3-ylvinyl)pyrimidine or a pharmaceutically acceptable salt thereof.

12. The method of claim 1, wherein the method further comprises administration of one or more additional therapeutic agent.

13. A method for enhancing colonic motility comprising administration of (R)-5-((E)-2-pyrrolidin-3-ylvinyl)pyrimidine or a pharmaceutically acceptable salt thereof.

14. A method for treating a mammal in need thereof with (R)-5-((E)-2-pyrrolidin-3-ylvinyl)pyrimidine or a pharmaceutically acceptable salt thereof to relieve constipation.

15. The method of claim 14, wherein the mammal is an animal.

16. The method of claim 15, wherein the animal is a rabbit, cat, dog, cow, or horse.

17.-32. (canceled)
